(19) United States
(12) Patent Application Publication
(10) Pub. No.: US 2025/0032138 A1
(43) Pub. Date: Jan. 30, 2025

(54) ACOUSTIC TRANSDUCER AND METHOD OF FORMING THE SAME

(71) Applicant: Agency for Science, Technology and Research, Singapore (SG)

(72) Inventors: Jihang Liu, Singapore (SG); Eldwin Jiaqiang Ng, Singapore (SG)

(21) Appl. No.: 18/837,110

(22) PCT Filed: Feb. 14, 2023
(86) PCT No.: PCT/SG2023/050077

§ 371 Date: Aug. 8, 2024

(30) Foreign Application Priority Data
Feb. 24, 2022 (SG) 10202201863W

(51) Int. Cl. A61B 17/22

(52) U.S. Cl. CPC A61B 17/2202

(57) ABSTRACT

An acoustic transducer that may include a substrate including a cavity extending from a surface of the substrate, where the substrate further includes one or more acoustic channels, each of the one or more acoustic channels having a depth extending from the surface of the substrate and a length, the length greater than the depth, extending at least partially around the cavity. The acoustic transducer may also include a layered arrangement suspended over the cavity. The one or more acoustic channels may be configured to include an acoustic medium having a specific acoustic impedance lower than a specific acoustic impedance of the substrate.

### CROSS-REFERENCE TO RELATED APPLICATION

This application claims the benefit of priority of Singapore application No. 10202201863W filed Feb. 24, 2022, the contents of it being hereby incorporated by reference in its entirety for all purposes.

### TECHNICAL FIELD

Various embodiments of this disclosure may relate to an acoustic transducer. Various embodiments of this disclosure may relate to a method of forming an acoustic transducer.

### BACKGROUND

Ultrasound has been widely used in medical and clinical applications for more than half a century. Ultrasound diagnostic imaging technologies are based on detecting reflected acoustic waves from boundaries with mismatched acoustic impedances, and have been widely used in vivo and in vitro for disease diagnosis. As micromachined ultrasonic transducers (MUTs) technologies are able to achieve high quality imaging with high resolution and real time monitoring, these technologies with low quality factor (Q factor), broad bandwidth, small size, low power consumption, high transmit and receive sensitivities are promising to replace conventional devices based on bulk Lead Zirconate Titanate (PZT) in the current medical market. There are two main types of MUTs, based on their transduction mechanisms: piezoelectric micromachined ultrasonic transducers (pMUTs) and capacitive micromachined ultrasonic transducers (cMUTs).

Another key application for ultrasound is for medical therapy, where the goal is to insonify a targeted localized area with ultrasound waves, which may be used to generate heat, cavitation, or pressure to produce effects on the body. Therapeutic ultrasound has been used to treat pain, improve drug delivery efficiency through the skin (transdermal delivery) with low-intensity pressure ultrasound.

High intensity ultrasound also has many clinical applications. FIG. 1A illustrates drug delivery enhanced by cavitation effect. Sonodynamic therapy (SDT) is based on the stable or inertial cavitation effect as shown in FIG. 1A, where ultrasound can be combined with microbubbles to change the permeability of cell membrane or tissue wall to achieve high efficacy topical drug delivery.

Ultrasound may also be used with specific sonosensitizers for tumor tissue elimination. With the help of insonation, a sonosensitizer would be activated and release a large amount of reactive oxygen (ROS) to facilitate the apoptosis of identified cancer cells. SDT can mediate cellular toxicity directly, so it is promising for cancer treatment in the upcoming future.

Ultrasonic shock waves have also played a crucial role for inertial cavitation with non-thermal effects. Sharp discontinuous shock waves involving a sudden high output pressure and density have been used in extracorporeal lithotripsy especially for stone diseases as shown in FIG. 1B. FIG. 1B shows a schematic representation of an ureteroscope insertion for kidney stone treatment; and (inset) ultrasonic imaging beam forming.

Some fatal vascular lesions such as thrombus, stroke, and myocardial infarction, are also important targets for ultrasound therapy. Several research teams have shown that there is a maximal efficacy to enhance the breakup of a clot combined with insonation, thrombolytic agents and microbubbles rather than through any single drug effect.

As shown above, due to the huge potential of ultrasound therapy in the medical and clinical applications, the demand for corresponding ultrasound devices has ballooned. Unlike medical imaging which requires high frequencies (>2 megaHertz or MHz) and large bandwidth for good image resolution, the requirements for ultrasound therapy are high output pressure, with relatively low frequencies (typically 1 MHz or below), with transducer bandwidth less of a concern for continuous application. It is also important for therapeutic ultrasound to be applied at a localized region, and this can usually be better achieved with the transducer placed as close to the desired region—ideally on the tip of catheters or needles. This introduces a requirement of a low frequency transducer with an ultra-small packaging size that MUT technologies are especially well-suited for. The challenge then is to realize a high-pressure output within a small package size, particularly for in vivo applications. This would enable minimally invasive or non-invasive treatments for the local removal of tumour cells, stone crushing, and vascular clot breakup with high efficacy and high safety.

However, achieving high transmitter output within a small size is still challenging. Compared with traditional bulk PZT acoustic devices, MUTs operate in a flexural mode and can achieve a similar frequency, within a small size with comparable surface pressures. PMUTs are generally better suited for low frequencies (<1 MHz) compared to cMUTs, as the high-pressure output necessitates a large displacement—which may be difficult to achieve with conventional narrow-gap capacitive parallel plates without resorting to very high voltages across the cavity. PMUTs on the other hand, are actuated with a piezoelectric film, independent of the cavity depth. In order to increase the output pressures, the input voltage for the MUT is typically required to be increased accordingly. However, the disadvantage is the high voltages required and the huge power consumption. Hence, there is a need for increasing the transmit sensitivity for the MUT.

### SUMMARY

Various embodiments may relate to an acoustic transducer. The acoustic transducer may include a substrate including a cavity extending from a surface of the substrate, wherein the substrate further includes one or more acoustic channels, each of the one or more acoustic channels having a depth extending from the surface of the substrate and a length extending at least partially around the cavity. The length may be greater than the depth. The acoustic transducer may also include a layered arrangement suspended over the cavity. The one or more acoustic channels may be configured to include an acoustic medium having a specific acoustic impedance lower than a specific acoustic impedance of the substrate.

Various embodiments may include a method of forming an acoustic transducer. The method may include patterning a substrate such that the substrate includes a cavity extending from a surface of the substrate, wherein the substrate further includes one or more acoustic channels, each of the one or more acoustic channels having a depth extending from the surface of the substrate and a length extending at least partially around the cavity. The length may be greater than the depth. The method may also include forming a layered arrangement suspended over the cavity. The one or more acoustic channels may be configured to include an acoustic medium having a specific acoustic impedance lower than a specific acoustic impedance of the substrate.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1A illustrates drug delivery enhanced by cavitation effect.

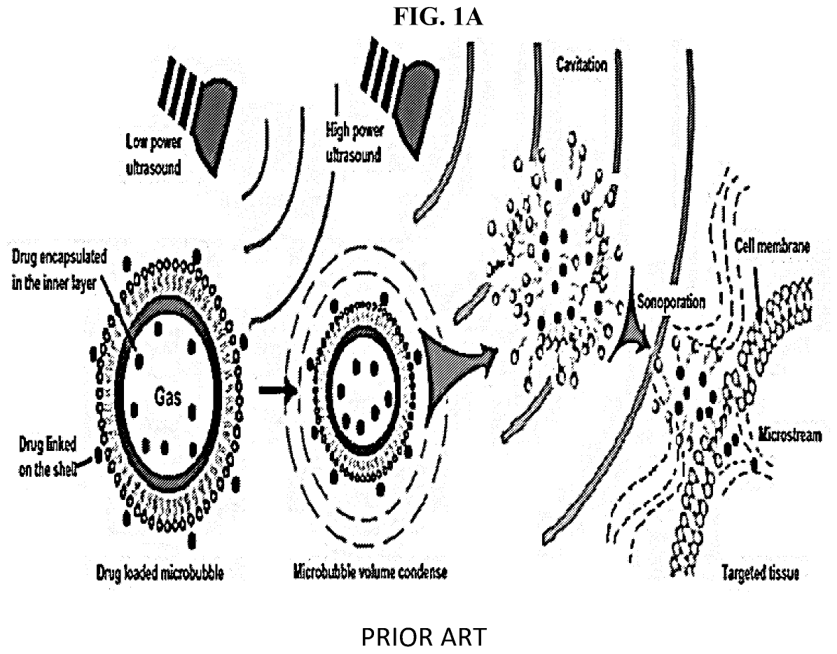

FIG. 1B shows a schematic representation of an ureteroscope insertion for kidney stone treatment; and (inset) ultrasonic imaging beam forming.

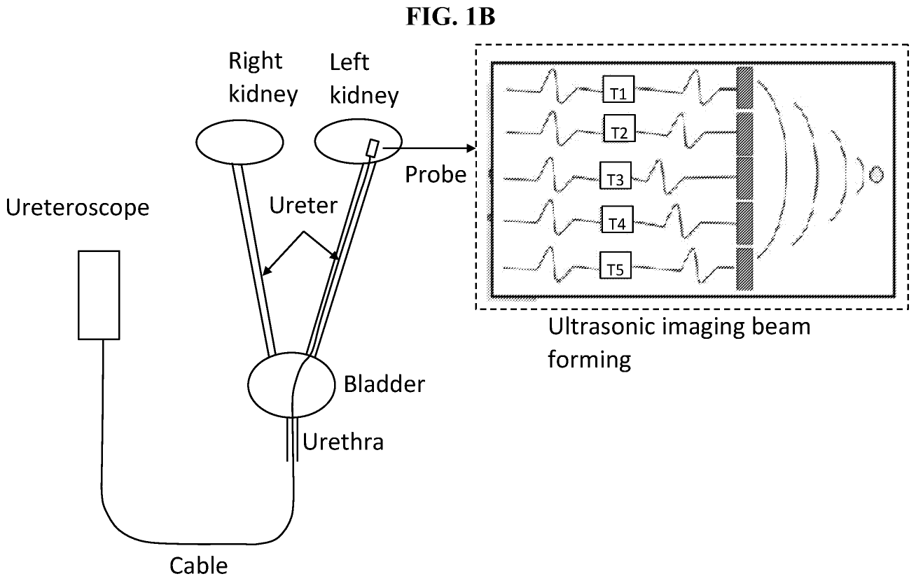

FIG. 2A illustrates piezoelectric micromachined ultrasonic transducers (pMUTs) with isolation trenches.

FIG. 2B shows a graph of axial location (micrometers or m) as a function of radical location (micrometers or m) illustrating deformation of the trench at resonant frequency.

FIG. 2C shows piezoelectric micromachined ultrasonic transducers (pMUTs) with etching holes.

FIG. 2D shows the thermo-elastic dissipation simulation results of the piezoelectric micromachined ultrasonic transducers (pMUTs) shown in FIG. 2C.

FIG. 3A shows a piezoelectric micromachined ultrasonic transducer (pMUT) with four linear holes.

FIG. 3B shows piezoelectric micromachined ultrasonic transducer (pMUT) with grooves inside and outside the cavity.

FIG. 4 is a schematic illustrating an acoustic transducer according to various embodiments.

FIG. 5 is a schematic illustrating a method of forming an acoustic transducer according to various embodiments.

FIG. 6A shows (left) a perspective view of an acoustic transducer according to various embodiments; and (right) a vertical cross-sectional view of the acoustic transducer according to various embodiments.

FIG. 6B shows a vertical cross-sectional view of the cavity type (or alternatively referred to as backside cavity type) acoustic transducer according to various embodiments.

FIG. 6C shows an operation of a capacitive micromachine ultrasonic transducer (cMUT) according to various embodiments.

FIG. 7 illustrates a conventional piezoelectric micromachined acoustic transducer (pMUT).

FIG. 8A is a block diagram illustrating the working concept of the acoustic transducer according to various embodiments.

FIG. 8B is a schematic illustrating the acoustic transducer and the working principle of the acoustic transducer according to various embodiments.

FIG. 9A illustrates the simulation set up of the acoustic transducer according to various embodiments.

FIG. 9B shows the parameters used in the simulation of the acoustic transducer according to various embodiments.

FIG. 9C illustrates the simulation set up of the acoustic transducer according to various embodiments in which the acoustic medium in the acoustic channel is the acoustic medium over the transducer stack.

FIG. 10A is a plot of pressure (Pressure Mag, in Pascals or Pa) as a function of frequency (freq, in Hertz or Hz) illustrating the transmit sensitivity of the conventional piezoelectric micromachined acoustic transducer (pMUT).

FIG. 10B is a plot of pressure (Pressure Mag, in Pascals or Pa) as a function of frequency (freq, in Hertz or Hz) illustrating the transmit sensitivity of an acoustic transducer with acoustic channel filled with a liquid according to various embodiments.

FIG. 11A is a plot of voltage (Voltage Mag, in millivolts or mV) as a function of frequency (freq, in Hertz or Hz) illustrating the receive sensitivity of the conventional piezoelectric micromachined acoustic transducer (pMUT).

FIG. 11B is a plot of voltage (Voltage Mag, in millivolts or mV) as a function of frequency (freq, in Hertz or Hz) illustrating the receive sensitivity of the acoustic transducer with acoustic channel filled with a liquid according to various embodiments.

FIG. 11C is a plot of impedance magnitude (in ohms or Ω)/impedance phase (in degrees) as a function of frequency (freq, in Hertz or Hz) illustrating the variation of the acoustic impedance magnitude and phase of the conventional piezoelectric micromachined acoustic transducer (pMUT).

FIG. 11D is a plot of impedance magnitude (in ohms or Ω)/impedance phase (in degrees) as a function of frequency (freq, in Hertz or Hz) illustrating the variation of the acoustic impedance magnitude and phase of the acoustic transducer with acoustic channel filled with a liquid according to various embodiments.

FIG. 12A shows a plot of displacement (in micrometers or μm) as a function of membrane radial coordinates (in micrometers or μm) illustrating the surface displacement magnitude for the conventional piezoelectric micromachined acoustic transducer (pMUT).

FIG. 12B shows a plot of displacement (in micrometers or μm) as a function of membrane radial coordinates (in micrometers or μm) illustrating the surface displacement magnitude for the acoustic transducer with acoustic channel filled with a liquid according to various embodiments.

FIG. 13 is a table showing the simulation results of a conventional device, the liquid-filled device according to various embodiments, the soft-solid PI device according to various embodiments, and the soft-solid PDMS device according to various embodiments.

FIG. 14A shows an acoustic transducer having a square horizontal cross-sectional shape according to various embodiments.

FIG. 14B shows an acoustic transducer having a circular horizontal cross-sectional shape according to various embodiments.

FIG. 14C shows an acoustic transducer having an annular horizontal cross-sectional shape according to various embodiments.

FIG. 14D shows an acoustic transducer having a pentagonal horizontal cross-sectional shape according to various embodiments.

FIG. 15 shows (a) an acoustic transducer having a continuous acoustic channel forming a square shape according to various embodiments; (b) an acoustic transducer having a discontinuous acoustic channel (i.e. a plurality of acoustic channels) forming a square shape according to various embodiments; (c) an acoustic transducer having a continuous acoustic channel forming a pentagonal shape according to various embodiments; (d) an acoustic transducer having a continuous acoustic channel forming a hexagonal shape according to various embodiments; (e) an acoustic transducer having a continuous acoustic channel forming a circular shape according to various embodiments; and (f) an acoustic transducer having a discontinuous acoustic channel (i.e. a plurality of acoustic channels) forming a circular shape according to various embodiments.

FIG. 16A is a plot of transmit pressure (in Pascals or Pa) as a function of frequency (in megahertz or MHz) showing the initial signal, the output signal with the isolation feature and the output signal without the isolation feature.

FIG. 16B is a schematic showing an acoustic transducer with an isolation trench.

FIG. 16C is a plot of transmit pressure (in Pascals or Pa) as a function of frequency (in megahertz or MHz) showing the initial signal, the output signal with the impedance matching layer and the output signal without the impedance matching layer.

FIG. 16D shows (left) a plot of transmit pressure (in Pascals or Pa) as a function of frequency (in megahertz or MHz) showing the initial signal; (middle) a plot of magnification as a function of frequency (in megahertz or MHz) showing the resonant channel gain factor according to various embodiments; and (right) a plot of transmit pressure (in Pascals or Pa) as a function of frequency (in megahertz or MHz) showing the initial signal, the output signal with resonant channel amplification according to various embodiments and the output signal without resonant channel amplification.

FIG. 17 is a plot of gain as a function of relative frequency shift (as a percentage (%) of the resonant frequency of the transducer unit) between the resonant frequency of the transducer unit of the acoustic transducer and the resonant frequency of the one or more acoustic channels of the acoustic transducer according to various embodiments.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a” “an” and “the” as used with regard to a feature or element include a reference to one or more of the features or elements.

In the context of various embodiments, the term “about” or “approximately” as applied to a numeric value encompasses the exact value and a reasonable variance, e.g. within 10% of the specified value.

As used herein, the term “and/or” includes any and all combinations of one or more of the associated listed items.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

Embodiments described in the context of one of the acoustic transducers are analogously valid for the other acoustic transducers. Similarly, embodiments described in the context of a method are analogously valid for an acoustic transducer, and vice versa.

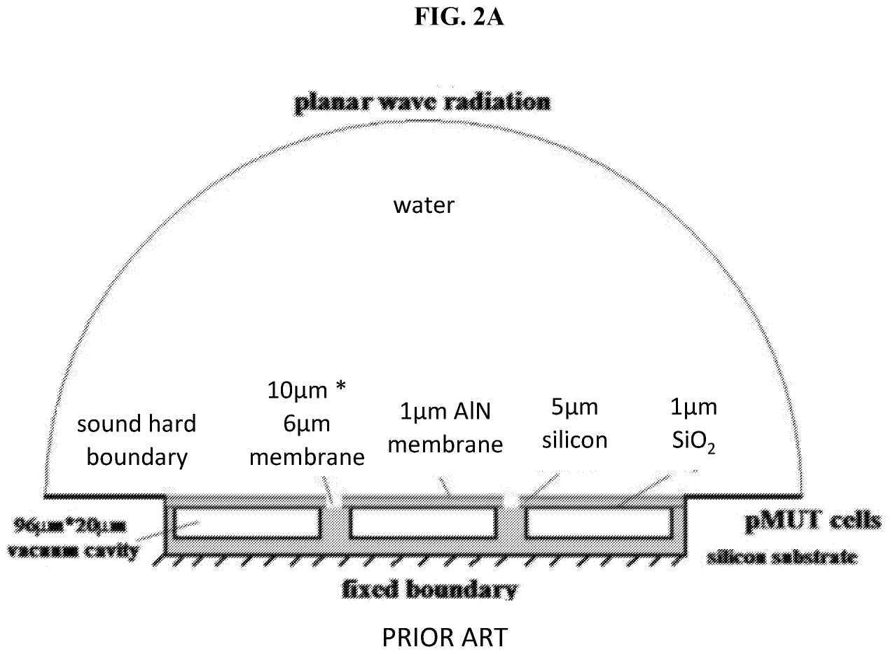

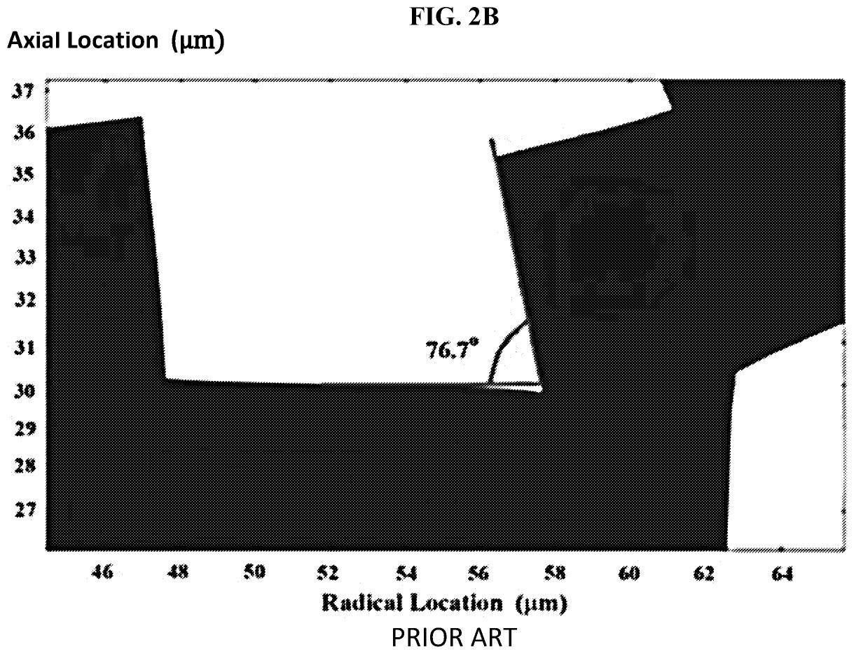

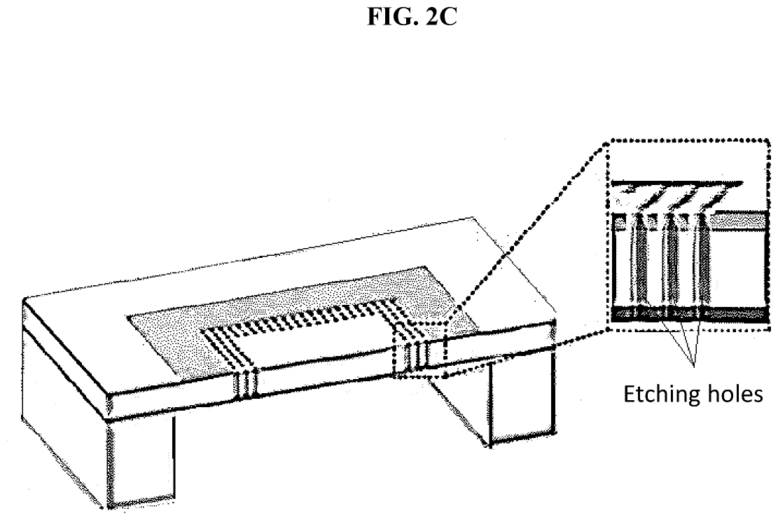

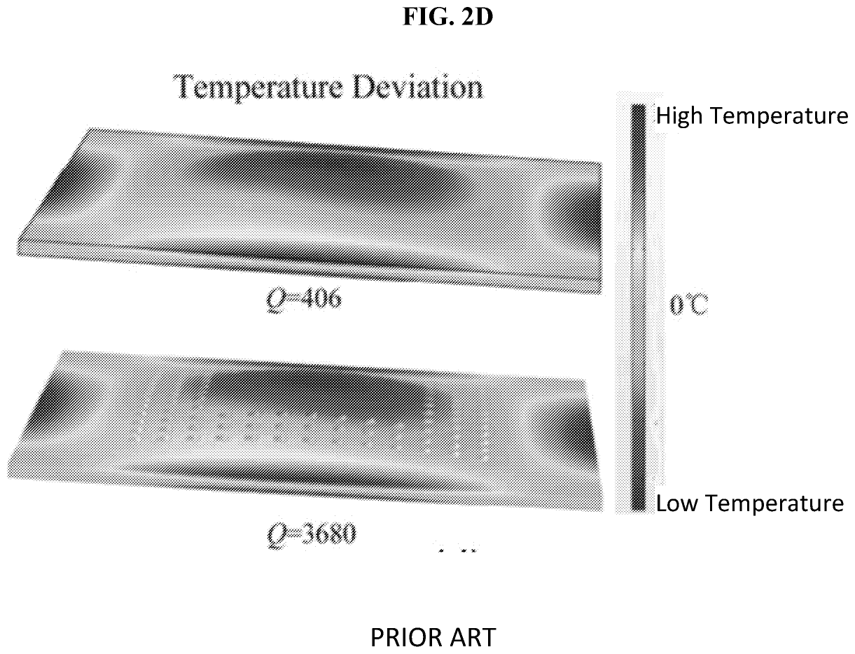

Several methods for transmission enhancement have been discussed in literature: isolation trenches have been designed to reduce the crosstalk between each pMUTs unit as shown in FIG. 2A, at the same time it can help increase the effective vibration area as shown in FIG. 2B. FIG. 2A illustrates piezoelectric micromachined ultrasonic transducers (pMUTs) with isolation trenches. FIG. 2B shows a graph of axial location (micrometers or m) as a function of radical location (micrometers or m) illustrating deformation of the trench at resonant frequency. Another possibility is to reduce the thermoelastic dissipation and increase the quality factor (Q factor) of air-coupled pMUTs. FIG. 2C shows piezoelectric micromachined ultrasonic transducers (pMUTs) with etching holes. FIG. 2D shows the thermo-elastic dissipation simulation results of the piezoelectric micromachined ultrasonic transducers (pMUTs) shown in FIG. 2C. A series of etching holes were placed around the effective vibration area as shown in FIG. 2C and there is a performance gain achieved in the simulation results as shown in FIG. 2D. However, for immersion-coupled pMUTs for medical applications, thermoelastic dissipation is not the main loss mechanism.

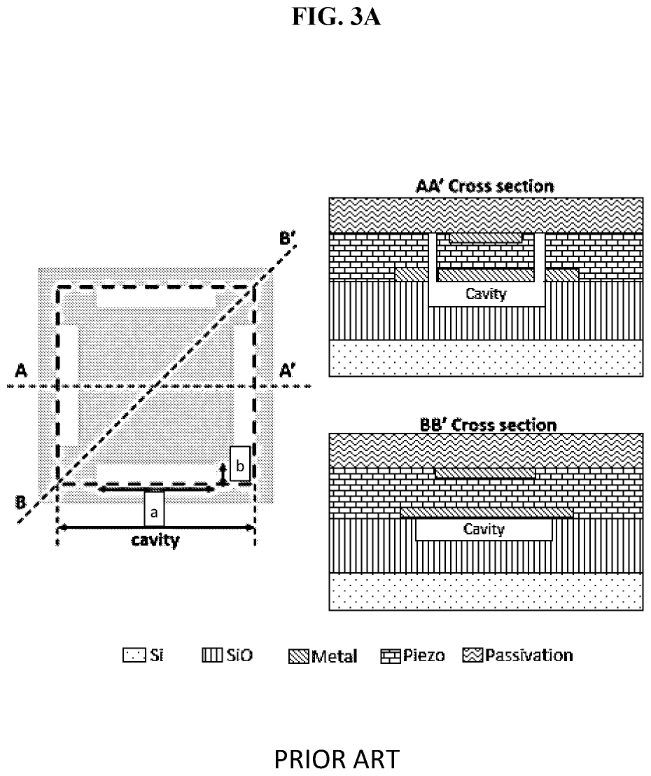

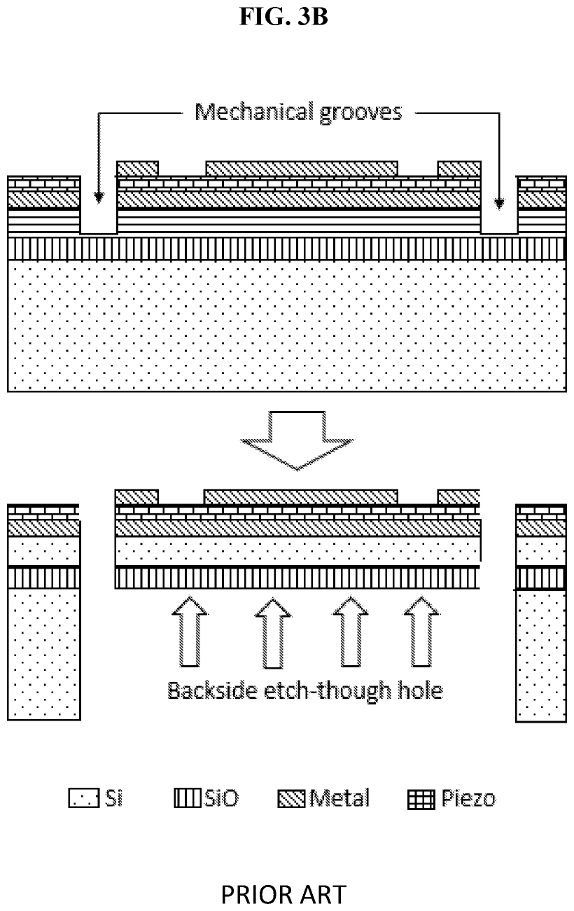

Another demonstrated concept was to use a pMUT with four rectangular holes that are sealed by the passive layer, allowing for an increase in the displacement and providing the capability to work in liquid environment as shown in FIG. 3A. FIG. 3A shows a piezoelectric micromachined ultrasonic transducer (pMUT) with four linear holes. The dimension of the holes was optimized to increase the pMUT displacement at least twice in relation with the regular clamped device.

A similar concept is as shown in FIG. 3B, grooves adjacent to annular and circular electrode pMUTs can help to reduce stress and increase the vibration area of the membrane. FIG. 3B shows piezoelectric micromachined ultrasonic transducer (pMUT) with grooves inside and outside the cavity. In the range of 10 kHz-70 kHz, the emission sound pressure level of the optimum AC-pMUT with grooves can be improved in the range of 2.8 dB-28.8 dB. In summary, the partial free-boundary condition is also an effective way to enhance the Q factor and transmission sensitivity.

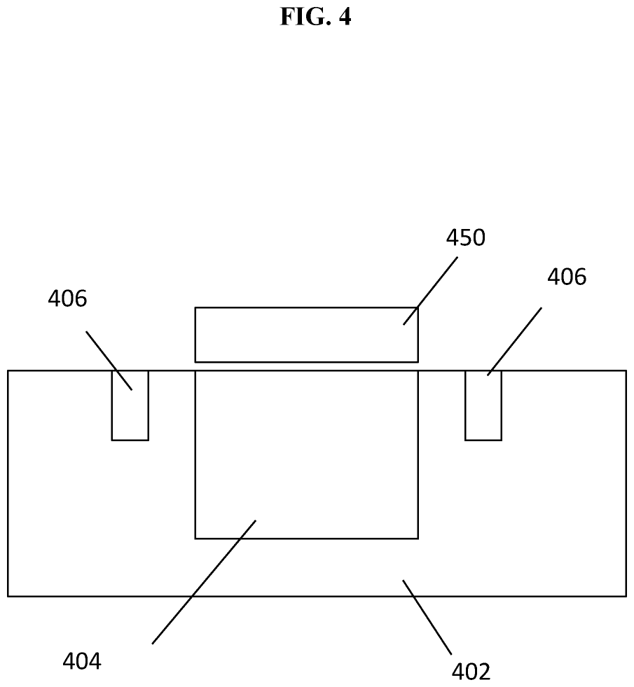

FIG. 4 is a schematic illustrating an acoustic transducer according to various embodiments. The acoustic transducer may include a substrate 402 including a cavity 404 extending from a surface of the substrate, wherein the substrate 402 further includes one or more acoustic channels 406, each of the one or more acoustic channels 406 having a depth extending from the surface of the substrate 402, and a length extending at least partially around the cavity 404. The length may be greater than the depth. The acoustic transducer may further include a layered arrangement 450 suspended over the cavity 404. The one or more acoustic channels 406 may be configured to include an acoustic medium having a specific acoustic impedance lower than a specific acoustic impedance of the substrate 402.

In other words, the acoustic transducer may include a substrate 402 which has been patterned to define a cavity 404 as well as one or more acoustic channels 406. The cavity 404 and the one or more acoustic channels 406 may have depths extending from a surface of the substrate 402. Additionally, the lengths of the one or more acoustic channels 406 may surround or partially surround an axis extending along the depth of the cavity. The acoustic transducer may include a layered arrangement 450 above the cavity 404. The one or more acoustic channels 406 may be configured to carry or hold an acoustic medium which has a specific acoustic impedance lower than a specific acoustic impedance of the substrate 402.

For avoidance of doubt, FIG. 4 seeks to illustrate some components of the acoustic transducer and is not intended to limit the shape, size, orientation, arrangement etc. of the components.

The one or more acoustic channels 406 carrying or holding the acoustic medium may act as a frequency dependent acoustic amplifier and boost the transmit function of a transducer unit including the layered arrangement 450 and the cavity 404. In other words, the one or more acoustic channels 406 with the acoustic medium may act as an additional resonant unit or part. The acoustic medium may be a portion of the resonant unit or part, and may influence the properties of the resonant unit or part. The acoustic transducer may be a multi-unit resonant acoustic transducer system. In various embodiments, the layered arrangement 450 may be surrounded by one or more resonant channel units formed by the one or more acoustic channels. These resonant channel units may function like a kind of frequency-dependent acoustic amplifier, which can boost the transmit function of the transducer unit including the layered arrangement 450 and the cavity 404.

The acoustic transducer may be configured to generate an output acoustic signal upon application of a voltage to the layered arrangement 450. The layered arrangement 450 may be configured to move or vibrate the layered arrangement 450 (e.g. via piezoelectric effect or capacitive electrostatic effect) upon application of the voltage to the layered arrangement 450, thereby generating the output acoustic signal. In various embodiments, e.g. in a piezoelectric micromachined ultrasonic transducer (pMUT), the layered arrangement 450 may include a membrane stack and a transducer stack over the membrane stack. The acoustic transducer may be configured to generate the acoustic signal upon application of the voltage to the transducer stack. The transducer stack may be configured to move or vibrate the layered arrangement 450 (e.g, via the piezoelectric effect) upon application of the voltage to the transducer stack, thereby generating the output acoustic signal. In various other embodiments, e.g. in a capacitive micromachined ultrasonic transducer (cMUT), the layered arrangement 450 may include or consist of a moveable electrode. The acoustic transducer may further include a fixed electrode. The acoustic transducer may be configured to generate the acoustic signal upon application of a potential difference between the moveable electrode and the fixed electrode. The moveable electrode may be configured to be deformed or moved upon application of the potential difference, thereby generating the output acoustic signal.

Additionally or alternatively, the acoustic transducer may be configured to generate an output electrical signal upon an ultrasound wave incident onto the layered arrangement 450. The layered arrangement 450 may be moved or vibrated upon the ultrasound wave incident on the layered arrangement 450, thereby moving the layered arrangement 450 to generate the output electrical signal (e.g. via converse piezoelectric effect or capacitive effect). For instance, in various embodiments, e.g. in a pMUT, the acoustic transducer may be configured to generate an output electrical signal upon an ultrasound wave incident onto the membrane stack. The membrane stack may be moved or vibrated upon the ultrasound wave incident onto the membrane stack, thereby moving the transducer stack to generate the output electrical signal, e.g. via the converse piezoelectric effect. In various other embodiments, e.g. in a CMUT, the acoustic transducer may be configured to generate an output electrical signal upon an ultrasound wave incident onto the moveable electrode. The movable electrode may deform or move upon the ultrasound wave incident onto the moveable electrode, thereby generating the output electrical signal, e.g. via the capacitive effect.

The one or more acoustic channels 406 including the acoustic medium may provide amplification to an initial acoustic transmit signal generated by the layered arrangement 450 (upon application of the voltage to the layered arrangement 450). Additionally or alternatively, the one or more acoustic channels 406 including the acoustic medium may provide amplification to an initial electrical signal generated by the layered arrangement 450 (upon the ultrasound wave incident onto the layered arrangement 450).

In various embodiments, the cavity 404 may extend from the first surface of the substrate 402 to a second surface of the substrate 402 opposite the first surface (referred to as backside etching type or backport type), while in various other embodiments, the cavity 404 may not extend to the second surface of the substrate 402 (referred to as backside cavity type or more simply as cavity type).

In various embodiments, the substrate 402 may include a beam wall to at least partially define the cavity 404 and the one or more acoustic channels 406, the beam wall separating the cavity 404 from the one or more acoustic channels 406. The beam wall may be between the cavity 404 and the one or more acoustic channels 406. The beam wall may separate the one or more acoustic channels 406 from the cavity 404.

The beam wall may also act as an additional resonant unit or part. In various embodiments, the layered arrangement 450, the one or more acoustic channels 406 and the beam wall may be coupled with one another to provide amplification.

In various embodiments, an end portion of the beam wall may be in contact with the membrane stack 408. An opposing end portion of the beam wall may extend from the rest of the substrate 402.

In various embodiments, the beam wall may be configured to vibrate in a flexural mode upon application of an excitation voltage.

Various embodiments may have two or more resonant units or parts. Various embodiments may include the layered arrangement 450 and the one or more acoustic channels 406 including the acoustic medium as resonant units or parts, while various other embodiments may include the layered arrangement 450, the one or more acoustic channels 406 including the acoustic medium and the beam wall as resonant units or parts. Various embodiments may have an ultimate transmit function much larger than a conventional acoustic transducer (i.e. one with only the layered arrangement and underlying cavity, i.e. without the channels).

In various embodiments, the one or more acoustic channels 406 including the acoustic medium and the beam wall may provide amplification to an initial acoustic transmit signal generated by the layered arrangement 450 (upon application of the voltage to the layered arrangement 450).

In various embodiments, the one or more acoustic channels 406 including the acoustic medium and the beam wall may provide amplification to an initial electrical signal generated by the layered arrangement 450 (upon the ultrasound wave incident onto the layered arrangement 450).

Various embodiments may have a gain in quality (Q) factor of any number from about 1 to about 300 times higher, e.g. about 2 to about 300 times higher (strong coupling effect) or about 1 to about 10 times higher (modest coupling effect) compared to a conventional acoustic transducer. Various embodiments may have an output sensitivity gain of any number from about 1 to about 100 times higher, e.g. about 2 to about 100 times higher (strong coupling effects) or about 1 to about 10 times higher (modest coupling effect) compared to a conventional acoustic transducer.

Various embodiments may have strong coupling effect gains where a relative frequency shift between a resonant frequency of the transducer unit (f_{MUT}), the transducer unit including the layered arrangement and the cavity, and a resonant frequency of the one or more acoustic channels (f_{Channel}) is within 30% (i.e. ±30%) of the resonant frequency of the transducer unit (f_{MUT}), and/or where a 6 dB bandwidth of the transducer unit BW_{MUT} and a 6 dB bandwidth of the one or more acoustic channels BW_{Channel} overlap more than 80%.

Various embodiments may have modest coupling effect gains where a relative frequency shift between a resonant frequency of the transducer unit (f_{MUT}), the transducer unit including the layered arrangement and the cavity, and a resonant frequency of the one or more acoustic channels (f_{Channel}) is more than 30% (i.e. ±30%) but within 60% (i.e. ±60%) of the resonant frequency of the transducer unit (f_{MUT}), and/or where a 6 dB bandwidth of the transducer unit BW_{MUT} and a 6 dB bandwidth of the one or more acoustic channels BW_{Channel} overlap more than 30% but less than 80%.

In various embodiments, the acoustic medium may be or may include a solid, e.g. polyimide (PI) or polydimethylsiloxane (PDMS).

In various embodiments, the acoustic medium may be or may include a liquid, e.g. water or alcohol.

An acoustic medium such as a liquid or a solid may be preferred over an acoustic medium such as a gas in order to provide high Q factor.

In various embodiments, the acoustic medium may have a specific acoustic impedance selected from a range from 0.5 MRayl (mega-Rayleighs) to 5 MRayl.

In various embodiments, the substrate 402 may include a suitable semiconductor material such as silicon or germanium. The substrate 402 may have a specific acoustic impedance higher than the acoustic medium. For instance, a silicon substrate may have a specific acoustic impedance of 19 MRayl.

In various embodiments, the acoustic medium in the one or more acoustic channels 406 may also be over the layered arrangement 450. In other words, the acoustic medium in the one or more acoustic channels 406 may be the same as the acoustic medium over the layered arrangement 450. For instance, the entire acoustic device may be immersed in an acoustic medium such as water. In various other embodiments, the acoustic medium in the one or more acoustic channels 406 may not be the same as the acoustic medium over the layered arrangement 450.

In various embodiments, the acoustic medium, e.g. water or alcohol, may be provided to the one or more acoustic channels 406 when the acoustic transducer is in operation. In various embodiments, the acoustic transducer or substrate 402 may include an inlet for directing the acoustic medium, e.g. water, into the one or more acoustic channels 406, e.g. when the acoustic transducer is to commence operation. In various embodiments, the acoustic transducer or substrate 402 may include an outlet for directing the acoustic medium out of the one or more acoustic channels 406, e.g. when the transducer is not in operation.

In various embodiments, the one or more acoustic channels 406 may include the acoustic medium, e.g. PDMS, when the acoustic transducer is in operation and also when the acoustic transducer is not in operation.

In various embodiments, an effective stiffness of the acoustic medium may be lower than an effective stiffness of the layered arrangement 450 and an effective stiffness of the substrate 402. An acoustic medium with low stiffness may advantageously not significantly affect the vibration of the layered arrangement.

In various embodiments, the depth of each of the one or more acoustic channels may further extend through the layered arrangement 450. For instance, the depth of each of the one or more acoustic channels may further extend through the membrane stack and the transducer stack.

In various embodiments, the acoustic transducer may be a piezoelectric micromachined acoustic transducer (pMUT). As mentioned above, the layered arrangement 450 of the pMUT may include a transducer stack and a membrane stack. The transducer stack may include a first electrode, a second electrode, and a piezoelectric layer between the first electrode and the second electrode.

In various other embodiments, the acoustic transducer may be a capacitive micromachined acoustic transducer (cMUT). As mentioned above, the layered arrangement of the cMUT may include or consist of a moveable electrode. The acoustic transducer may further include a fixed electrode, i.e. the fixed electrode is not moved when the layered arrangement 450 is moved or vibrated. In various embodiments, the fixed electrode may be included or be part of the substrate 402. The fixed electrode may be an electrically conductive film attached to the substrate 402. In various embodiments, the fixed electrode may be the substrate 402.

In various embodiments, the layered arrangement 450, i.e. the membrane stack of the layered arrangement 450, may include a first dielectric layer, a second dielectric layer, and a membrane layer between the first dielectric layer and the second dielectric layer.

In various embodiments, the transducer stack may have a (horizontal) cross-sectional shape of any suitable shape.

In various embodiments, the transducer stack may have a (horizontal) cross-sectional shape selected from a group consisting of a square, a circle, an annulus, a polygon (e.g. a pentagon or hexagon), and an ellipse.

In various embodiments, there may be a single continuous acoustic channel 406 extending around the cavity 404. In various other embodiments, there may be a discontinuous acoustic channel (i.e. a plurality of acoustic channels) extending around the cavity 404. In various embodiments, the one or more acoustic channels may form a channel window of any suitable shape. In various embodiments, the one or more acoustic channels 406 may form a shape selected from a group consisting of a square, a circle, a pentagon, and a hexagon.

Various embodiments may not rely on any acoustic impedance matching layer. The function of the impedance matching layer may be different from the acoustic channel.

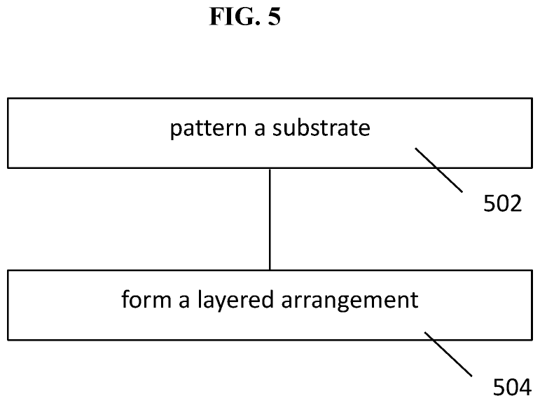

FIG. 5 is a schematic illustrating a method of forming an acoustic transducer according to various embodiments. The method may include, in 502, patterning a substrate such that the substrate includes a cavity extending from a surface of the substrate. The substrate may further include one or more acoustic channels, each of the one or more acoustic channels having a depth extending from the surface of the substrate and a length extending at least partially around the cavity. The length may be greater than the depth. The method may also include, in 504, forming a layered arrangement suspended over the cavity. The one or more acoustic channels may be configured to include an acoustic medium having a specific acoustic impedance lower than a specific acoustic impedance of the substrate.

In other words, the method may include forming a cavity and one or more acoustic channels on the substrate. The cavity and the one or more acoustic channels may have depths extending from a surface of the substrate. Additionally, the lengths of the one or more acoustic channels may surround or partially surround an axis extending along the depth of the cavity. The method may further include forming a layered arrangement.

For avoidance of doubt, FIG. 5 is intended to illustrate various steps according to various embodiments, and is not intended to limit the sequence of the various steps. For instance, step 504 may occur before, after or at the same time as step 502.

In various embodiments, the substrate may include a beam wall to at least partially define the cavity and the one or more acoustic channels, the beam wall separating the cavity from the one or more acoustic channels. The method may include patterning the substrate such that the substrate includes the beam wall.

In various embodiments, an end portion of the beam wall may be in contact with the membrane stack.

In various embodiments, the beam wall may be configured to vibrate in a flexural mode upon application of an excitation voltage.

In various embodiments, the acoustic medium may be a solid such as polyimide or polydimethylsiloxane. In various other embodiments, the acoustic medium may be a liquid such as water or alcohol.

In various embodiments, an effective stiffness of the acoustic medium may be lower than an effective stiffness of the layered arrangement and an effective stiffness of the substrate.

In various embodiments, the depth of each of the one or more acoustic channels may further extend through the layered arrangement.

In various embodiments, the acoustic transducer may be a piezoelectric micromachined acoustic transducer (pMUT). Forming the layered arrangement may include forming a membrane stack, and forming a transducer stack over the membrane stack. The transducer stack may include a first electrode, a second electrode, and a piezoelectric layer between the first electrode and the second electrode.

In various other embodiments, the acoustic transducer may be a capacitive micromachined acoustic transducer (cMUT). Forming the layered arrangement may include forming the moveable electrode. The method may also include forming a fixed electrode.

In various embodiments, the transducer stack may have a (horizontal) cross-sectional shape selected from a group consisting of a square, a circle, an annulus, a polygon (e.g. a pentagon or hexagon), and an ellipse.

In various embodiments, there may be a single continuous acoustic channel extending around the cavity. In various other embodiments, there may be a plurality of discontinuous acoustic channels extending around the cavity. In various embodiments, the one or more acoustic channels may form a channel window of any suitable shape. In various embodiments, the one or more acoustic channels may form a shape selected from a group consisting of a square, a circle, a pentagon, and a hexagon.

Various embodiments may relate to a method of operating an acoustic transducer as described herein. The method may include providing a voltage to a transducer stack of the acoustic transducer to generate an output acoustic signal. The method may additionally or alternatively include providing an ultrasound wave to the membrane stack of the acoustic transducer to generate an output electrical signal.

Various embodiments may relate to an acoustic transducer, e.g. a MUT structure, differentiated from the conventional MUT by containing three coupled resonant units involving a structural MUT (i.e. transducer stack) with underlying cavity, a beam wall, and an acoustic channel. Each of these units may have their own resonant frequencies. At particular resonant frequencies, there may be a large amplitude movement resulting in a huge output pressure. The mechanism of the multi-resonant unit coupling system concept is described in detail later. COMSOL simulation is carried out for one implementation in which the acoustic channel is filled with liquid and another implementation in which the acoustic channel is filled with a soft solid. While pMUTs are discussed in more detail herein, various embodiments may include or relate to cMUTs or other types of membrane acoustic transducers. There may be huge performance gains after optimization, and the gains may be critical for the medical and clinical applications described above which require high transmit sensitivity, high receive sensitivity and ultra-small needle size packaging.

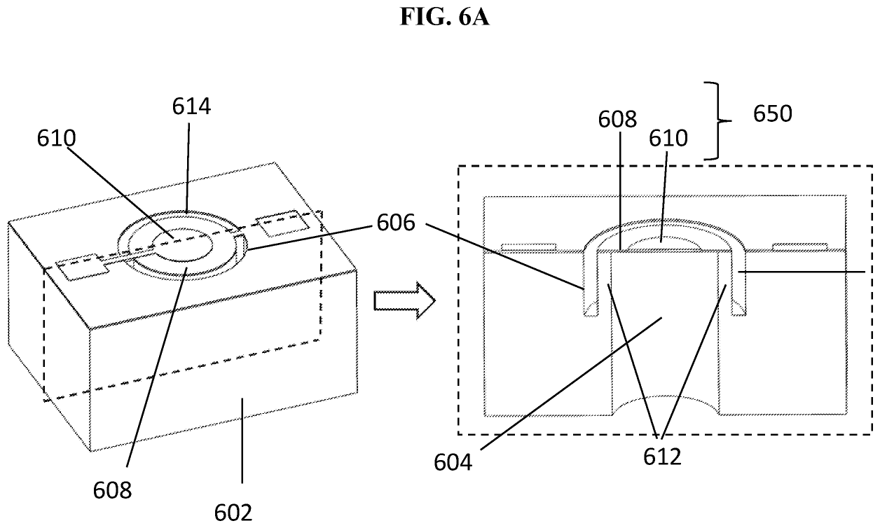

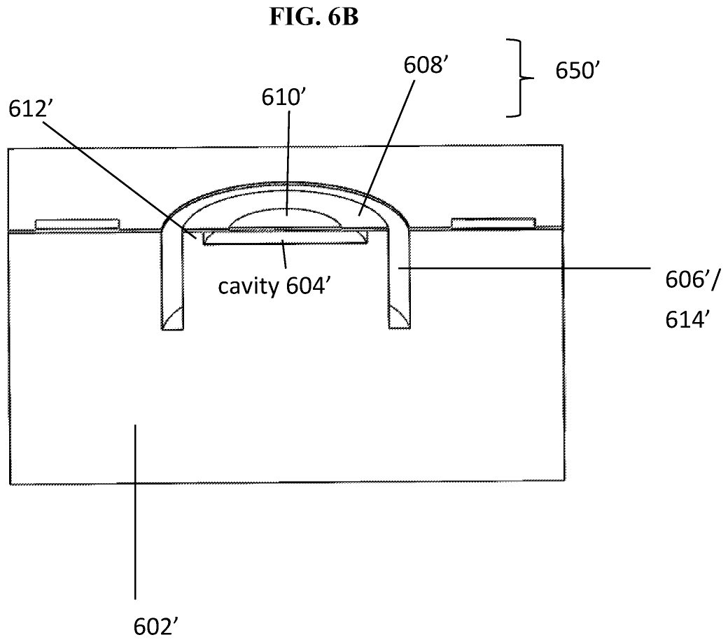

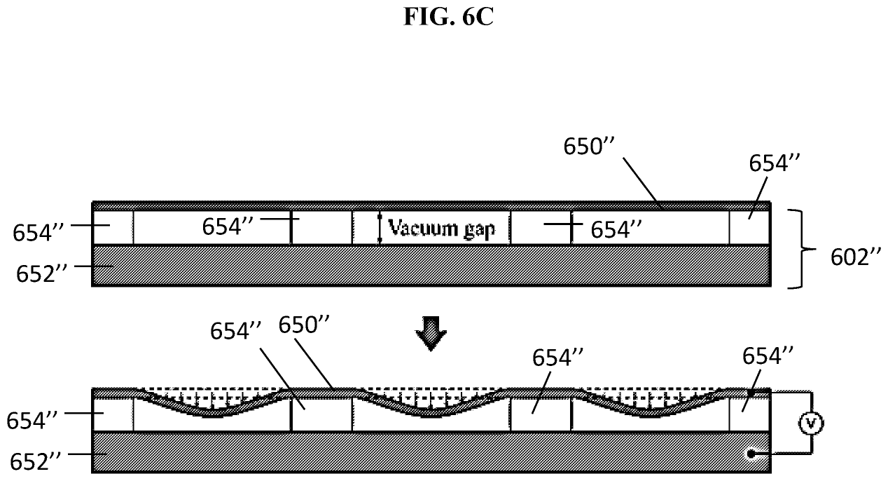

FIG. 6A shows (left) a perspective view of an acoustic transducer according to various embodiments; and (right) a vertical cross-sectional view of the acoustic transducer according to various embodiments.

The acoustic transducer may be a multi-unit resonant coupling system including three resonant units. The first resonant unit may be a piezoelectric transducer stack 610 located in the middle of the die with underlying cavity. The acoustic transducer may be a back port type or a cavity type. FIG. 6A shows the back port type (alternatively referred to as backside etching type) with the cavity 602 extending from the first surface of the substrate 602 to the second surface of the substrate opposite the first substrate. The second resonant unit may be a deep acoustic channel 606, which surrounds the center piezoelectric transducer stack 610. The piezoelectric transducer stack 610 may be over the membrane stack 608, which may be over the substrate 602. The piezoelectric transducer stack 610 and the membrane stack 608 may make up the layered arrangement 650. For the “liquid-filled” implementation, the acoustic channel 606 may be filled with an acoustic medium 614, such as water or liquid. For the “soft-solid-filled” implementation, the acoustic channel may instead include a soft solid material with low Young's modulus, such as PDMS or polyimide. The specific acoustic impedance of the acoustic medium 614 may be significantly lower than that of the material of the substrate 602. The third resonant unit may be the acoustic beam wall 612, which may be formed at the same time as the acoustic channel etch. Each of these units may be operated in a frequency range where the vibration of one mode may dominate, or where the vibration of the mode may be coupled synergistically, such that two or three resonant parts may be coupled, depending on the different design parameters. The concept of resonant units coupling effect may be described later herein. FIG. 6B shows a vertical cross-sectional view of the cavity type (or alternatively referred to as backside cavity type) acoustic transducer according to various embodiments. The cavity 604′ may not extend from the first surface of the substrate 602′ to the second surface of the substrate 602′ opposite the first surface. Instead, the cavity 604′ may be enclosed by the substrate 602 (including beam wall 612′) and the membrane stack 608′. In addition, the acoustic transducer may include the acoustic channel 606′ including acoustic medium 614′. The acoustic transducer may also include transducer stack 610′ over the membrane stack 608′. The transducer stack 610′ and the membrane stack 608′ may make up the layered arrangement 650′.

FIG. 6C shows an operation of a capacitive micromachine ultrasonic transducer (cMUT) according to various embodiments. The acoustic channels are not shown in FIG. 6C. The acoustic transducer may include a substrate 602″, which may be a fixed electrode 652″; and a supporting layer 654″ on the fixed electrode 652″. The substrate 602″ may include an electrical conductor such as doped silicon. It may also be envisioned that in other embodiments, the fixed electrode may be another component, e.g. an electrically conductive film, attached to the substrate. The acoustic transducer may include a layered arrangement 650″, which may consist of a single moveable electrode. The moveable electrode may be spaced from the fixed electrode 652″ by the supporting layer 654″. The supporting layer 654″ may include an insulator. During operation, the movable electrode may move or be deformed, while the fixed electrode 652″ may remain unchanged/unmoved.

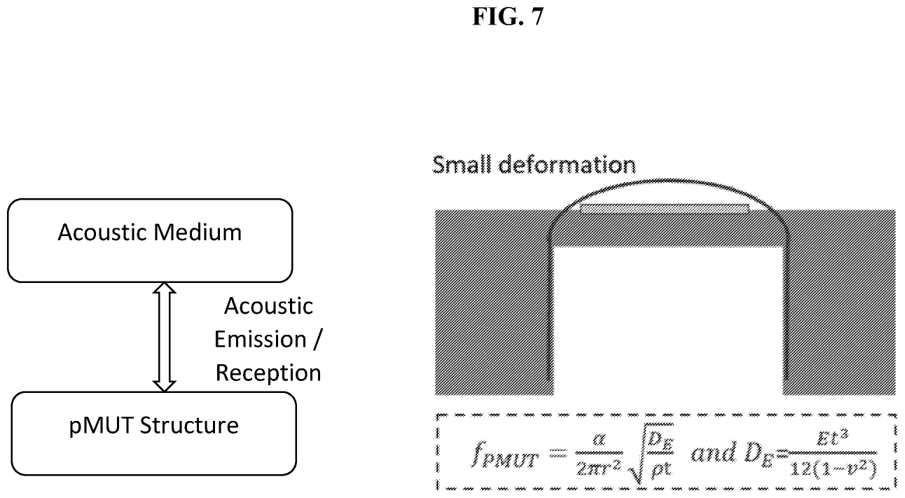

FIG. 7 illustrates a conventional piezoelectric micromachined acoustic transducer (pMUT). The core of the conventional pMUT is a layered piezoelectric stack, which includes the bottom electrode, piezoelectric layer, and the top electrode. The stack may be attached to a membrane structural layer, another piezoelectric stack, or have the electrodes of different stiffness/thickness. When the piezoelectric layer is driven by a time-varying electrical signal, the membrane will vibrate up and down vertically. For actuation, a part of the electrical energy transforms into kinetic energy due to the reverse piezoelectric effect. The pMUT may also be operated as a sensor by receiving an acoustic pressure that is then converted into an electrical signal by the piezoelectric effect.

In practical applications, pMUT device is usually coupled to air or water or even body tissue, which could be defined as the surrounding acoustic medium where the acoustic waves propagate into. As the membrane vibrates, the surrounding acoustic medium would be compressed. The relationship between them can be simplified as shown in FIG. 7, hence part of the kinetic energy would be transmitted into the acoustic medium. During this process, the pMUT can influence the surrounding acoustic medium, and in reverse, the surrounding medium can also influence the pMUT itself. A factor termed “transmit sensitivity” is used to describe the transmitting capability, which can be defined as the transmitted pressure divided by the input voltage. This parameter is influenced largely by both the pMUT and the properties of acoustic medium (density, acoustic impedance, viscoelasticity and etc.)

In the case of a conventional edge-clamped circular diaphragm with low intrinsic stress, the membrane behaves as a plate with the resonant frequencies f_{pMUT} given as shown in FIG. 7, where α is the resonance mode constant, r is the radius of the diaphragm, D_{E} is the flexural rigidity, ρ is the effective density of the diaphragm, t is the diaphragm thickness, E is the effective Young's modulus, and ν is Poisson's ratio. Once the material and thickness are chosen, the most common way to design the expected frequency of the pMUT is to adjust the radius r of the diaphragm.

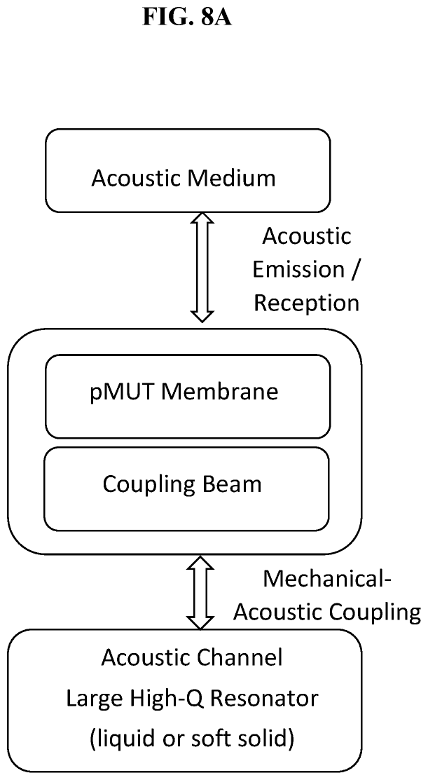

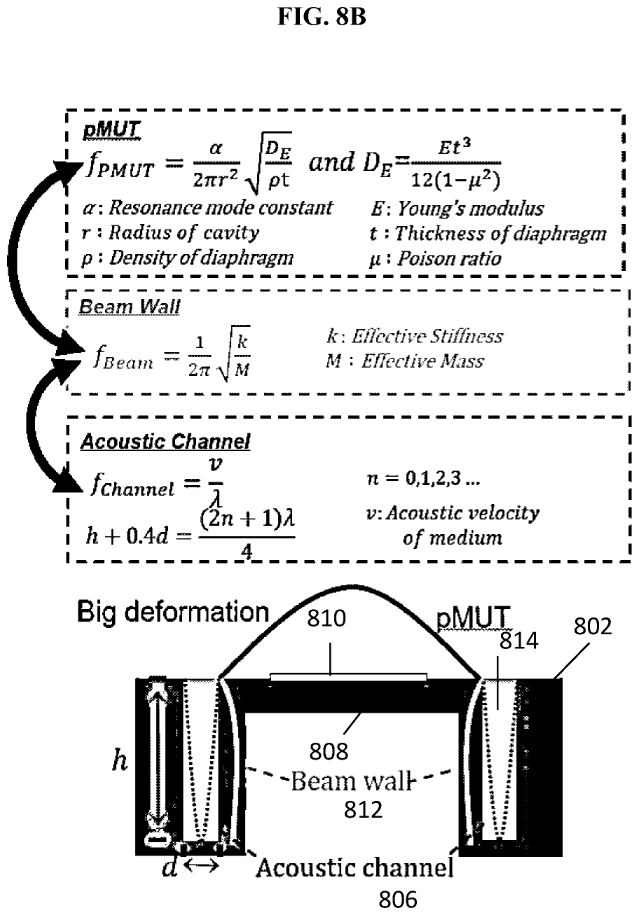

FIG. 8A is a block diagram illustrating the working concept of the acoustic transducer according to various embodiments. FIG. 8B is a schematic illustrating the acoustic transducer and the working principle of the acoustic transducer according to various embodiments.

The acoustic transducer may include the transducer stack 810, a beam wall 812 that vibrates in the lateral direction, and an acoustic channel 806. The beam wall 812 may be a type of resonant structure, similar to the transducer stack 810. The beam wall 812 may be fixed on one end to the substrate 802 and may be connected to the pMUT membrane 808 on the other. The beam wall 812 is tightly coupled to the transducer stack 810 and the resonant frequency of this beam-pMUT system is given in FIG. 8B. Since this is a tightly coupled system, the beam-pMUT system frequency f_{Beam-pMUT} may be dependent on the effective beam-pMUT mass and as well as the stiffness of the beam and pMUT. The effective stiffness k may be affected by the Young's modulus (E), the inertial moment (I) of the beam cross-section and the beam length (h). The expected frequency f_{Beam-pMUT} may be controlled by varying the geometry or materials of the beam and the pMUT.

The third resonant unit is a high Q acoustic channel resonator, which operates in a tube resonance mode. When the acoustic channel 806 is filled with an acoustic medium 814, such as liquid or a low-stiffness solid, an acoustic resonance may be induced in the material. The acoustic channel 806 may function as a resonator, and may operate in a tube resonance mode, such as the fundamental quarter-wavelength mode where the top part of the channel 806 in contact with the surrounding medium is in an open boundary condition, while the bottom part of the channel 806 bounded by the substrate 802 is in a fixed boundary condition. End-effects of the channel depth may also need to be accounted for designing the acoustic channel 806. The displacements in the acoustic medium 814 at resonance may be large at the top and small at the bottom (substrate side) of the channel 806.

The acoustic medium 814 in the channel 806 may have a specific acoustic impedance much smaller than that of the substrate 802, such that the acoustic waves in the acoustic medium 814 can be reflected at the channel-substrate interfaces and the acoustic energy is kept within the channel 806 and does not dissipate into the substrate 802. For example, if the substrate 802 is silicon with a specific acoustic impedance around 19 MRayl, the acoustic medium may be water, polydimethylsiloxane (PDMS), or various epoxies with specific acoustic impedances in the range of 0.5-5 MRayl. Solid materials may be advantageous over liquids in terms of device robustness. For the acoustic channel medium 814, a low stiffness may be preferred, with an effective stiffness of the acoustic channel 814 lower than that of the pMUT or substrate 802 so as not to significantly affect the pMUT vibration. The acoustic channel medium 814 may also be formulated to have low acoustic losses since the objective is to create a high quality factor (Q) acoustic channel resonator. In order to realize a high Q coupled pMUT system, the density of the acoustic channel medium 814 may need to be sufficiently high. Gases, for example, may not be well-suited as the effect of the coupled acoustic resonator would be minimal compared to the transducer stack 810 or beam wall 812. The acoustic channel 806 containing the acoustic medium 814 may therefore differ from an isolation trench. The objective of an isolation trench is to isolate the pMUT units in array to decrease the crosstalk between the pMUT units, and to cut the wave propagating path to let the wave energy stop and reflect back at the isolation trench interface. Hence, the bigger the acoustic impedance mismatch, the better the isolation effect will be. In order to maximize the isolation effect, the isolation trench should contain vacuum or air. In contrast, the acoustic channel 806 according to various embodiments may be used for resonating and coupling so the channel 806 may be filled with an acoustic medium 814 with a certain acoustic impedance. The channel 806 may not be in vacuum.

The resonant acoustic channel 806 may be a kind of passive resonator. The acoustic channel 806 may gain certain incident wave energy from the adjacent transducer stack 810. The acoustic medium 814 in the acoustic channel 806 may be the same as or may be different from the acoustic medium over the transducer stack 810. The acoustic medium 814 in the channel 806 should be chosen carefully to match the coupling condition specified.

The purpose of the acoustic channel 806 is to couple with the structural beam-pMUT to ultimately supply energy to increase the vibration and pressure of the system for a given input voltage amplitude. When the electrical energy from the driving electrical signal is converted into mechanical energy by the transducer stack 810, part of the energy goes into the transducer stack 810 and the acoustic medium 814, while part of it is stored in the acoustic channel 806, which may function as a resonator. For a given voltage amplitude signal, the lower-Q transducer stack 810 may vibrate and cause the tightly coupled beam wall 812 to vibrate at the same time. The beam wall 812 may then compress or pull against the acoustic channel 806. If the frequency of the actuation is well matched to the eigenmode frequency of the acoustic channel 806, a resonant (standing) acoustic wave may build up in the acoustic channel 806. This energy stored in the acoustic channel 806 may build up over time and the amplitude of the vibration may increase. The vibration of the transducer stack 810, and hence the acoustic pressure may thus increase. The resonant frequency of the acoustic channel f_{Channel} may be varied by the height h and the width d as well as the properties of channel filled acoustic medium 814.

The relationship between the resonant units can be simplified as shown in FIG. 8B. For the system with three resonant units, the maximum response may be derived when the frequencies of the resonant units are matched, e.g. f_{pMUT}≈f_{Beam}≈f_{Channel}. The transducer stack 810 and the beam wall 812 may couple directly through mechanical connection, while the beam wall 812 and the acoustic channel 806 may couple through the acoustic medium 814. The transducer stack 810 and the acoustic channel 806 may also couple through acoustic medium 814. As a result, the deformation of the transducer stack 810 may be amplified much larger than the stack in a conventional transducer, i.e. a conventional pMUT. At the same time, the transducer stack 810 may generate much more kinetic energy and transmit much more pressure into the acoustic medium 814.

Although a larger transmit sensitivity (pressure per input voltage) is obtained, it should be also noted that a higher Q also decreases the motional resistance, resulting in a higher current being drawn. However, there may be power efficiency benefits as the static capacitance of the acoustic transducer remains mostly unchanged, and the ratio of output acoustic energy to electrical energy required to charge the piezoelectric capacitor may increase. The lower voltage system may also be advantageous from the driving circuit perspective in terms of power efficiency and lower requirements for high voltages.

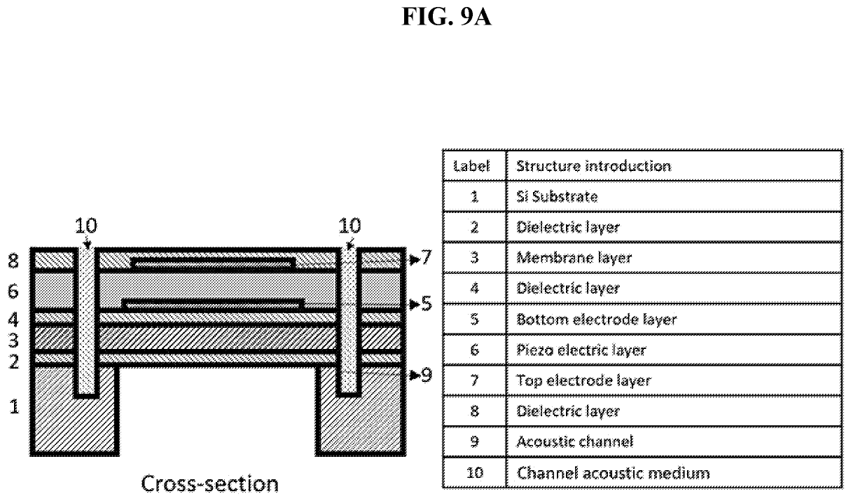

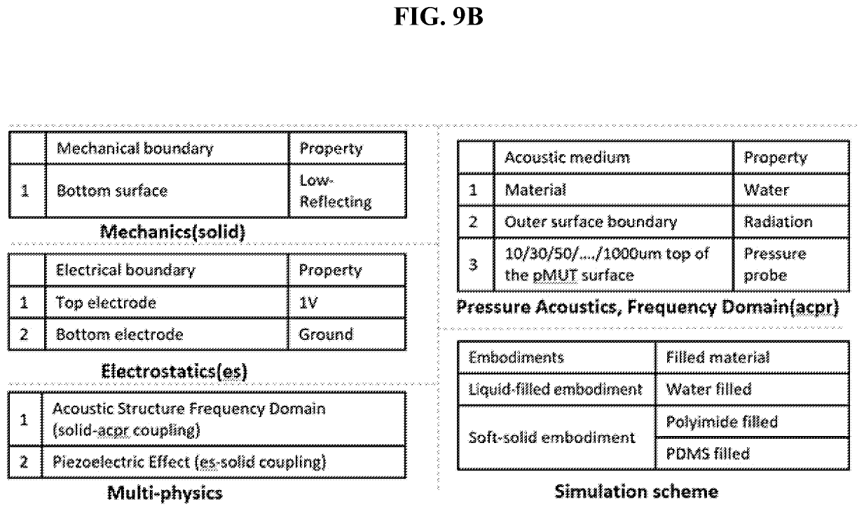

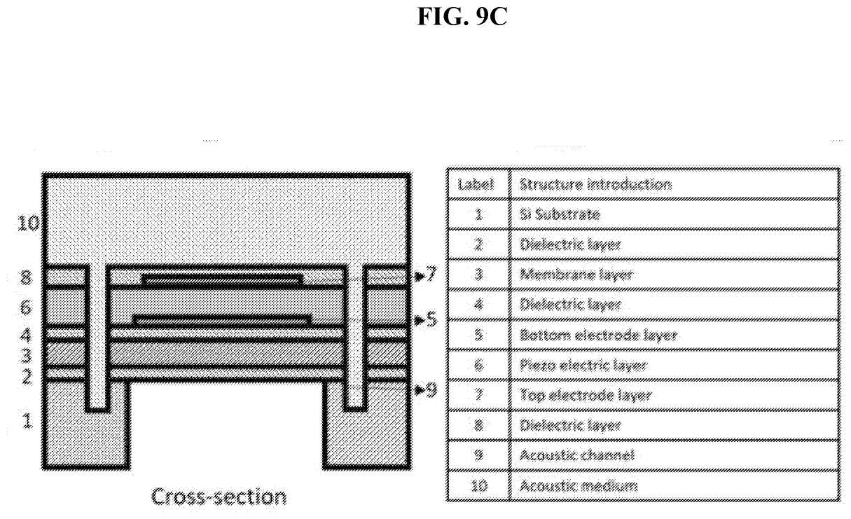

This concept was verified by COMSOL simulation. FIG. 9A illustrates the simulation set up of the acoustic transducer according to various embodiments. FIG. 9B shows the parameters used in the simulation of the acoustic transducer according to various embodiments. In this case, the simulation includes three main physics with two multi-physics coupling interfaces to simulate the pMUT device operation in water.

The first physics is solid mechanics field, which contains all the solid structures as shown in the cross-sectional view. It is usually used for static and dynamic solid mechanics solution. Just as described above, a piezoelectric stack of Top Electrode-Piezoelectric Layer-Bottom Electrode is stacked on a silicon (Si) membrane. Dielectric layers are designed and used for electrical isolation. The acoustic channel unit may be formed by Deep Reactive Ion Etching (DRIE) or wet etching after the last backside etching step. The beam wall unit is a natural result of the processing steps, and is in the middle of the other two resonant units. For comparison, both the proposed design (with acoustic channel) according to various embodiments and the conventional design (without acoustic channel) are simulated. The second physics is electrostatics, which is coupled with the structural condition to model the piezoelectric layer. To simulate the sensitivity, 1 volt is applied to the top electrode and the bottom electrode is grounded. The third physics is pressure acoustics frequency domain field, which is used to simulate the acoustic wave transmission in the acoustic medium. The solid and acoustic physics are coupled at the pMUT to the acoustic medium interface, while the solid mechanics and electrostatics are coupled for the piezoelectric layer.

For our proposed pMUT design according to various embodiments, two variants may be simulated. One variant is the liquid-filled implementation, where the acoustic channel is filled with water. The other variant is the soft-solid implementation, where the channel is filled with a soft material such as polyimide or PDMS.

In simulation, the TX sensitivity, RX sensitivity, impedance magnitude and phase, as well as the displacement properties of both the conventional pMUT and the proposed liquid-filled embodiment pMUT are obtained. The 3 MHz eigenfrequency pMUT with 60 μm radius cavity is selected here as an example. The difference between the conventional pMUT and the liquid-filled embodiment is the 90 μm optimal depth acoustic channel.

FIG. 9C illustrates the simulation set up of the acoustic transducer according to various embodiments in which the acoustic medium in the acoustic channel is the acoustic medium over the transducer stack.

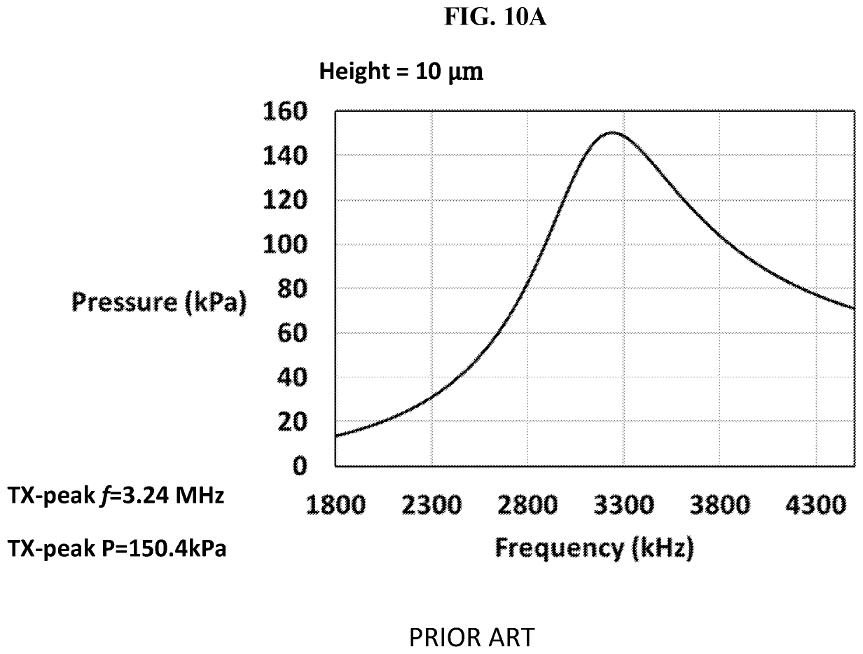

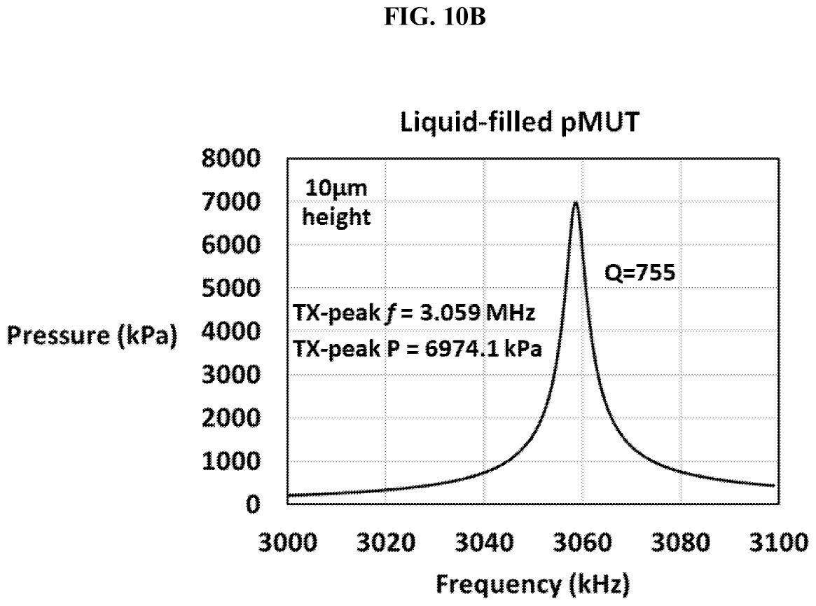

FIG. 10A is a plot of pressure (Pressure Mag, in Pascals or Pa) as a function of frequency (freq, in Hertz or Hz) illustrating the transmit sensitivity of the conventional piezoelectric micromachined acoustic transducer (pMUT). FIG. 10B is a plot of pressure (Pressure Mag, in Pascals or Pa) as a function of frequency (freq, in Hertz or Hz) illustrating the transmit sensitivity of an acoustic transducer with acoustic channel filled with a liquid according to various embodiments.

The transmit sensitivity (kPa/V) was measured near the surface of the pMUT, at a distance of 10 μm above of the pMUT surface. The peak value of the liquid-filled device according to various embodiments reaches 6974.1 kPa which is about 46 times higher than that of the conventional device (150.4 kPa). In addition, the Q factor increased by 209 times from 3.6 for the conventional device to 755 for the liquid-filled device according to various embodiments.

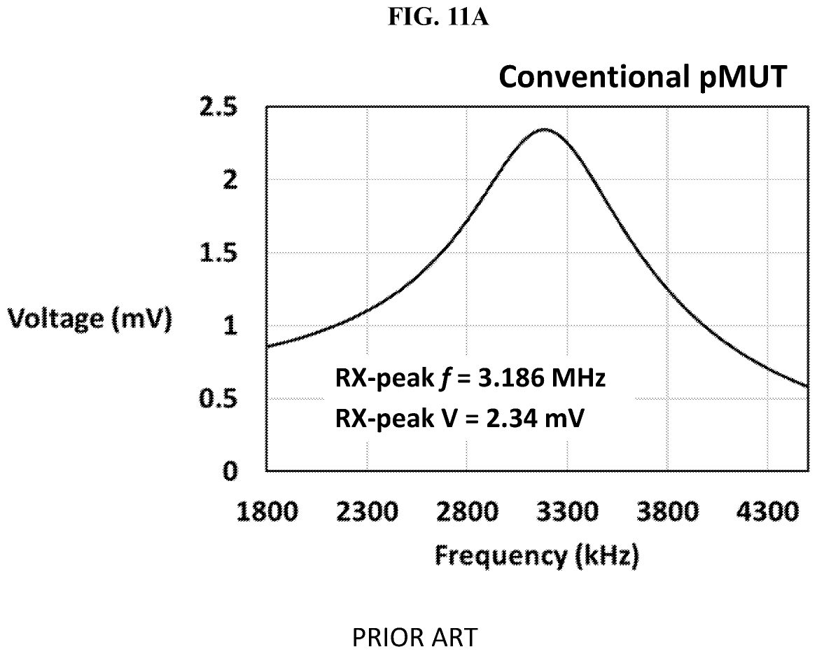

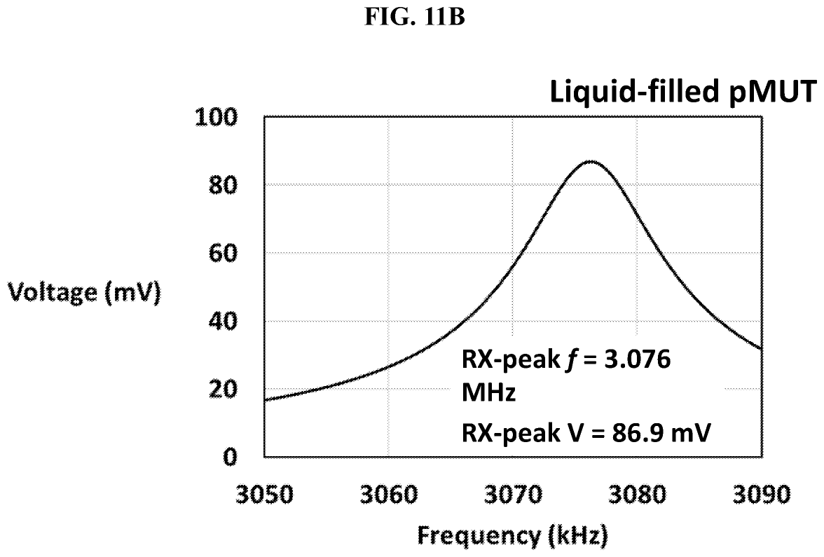

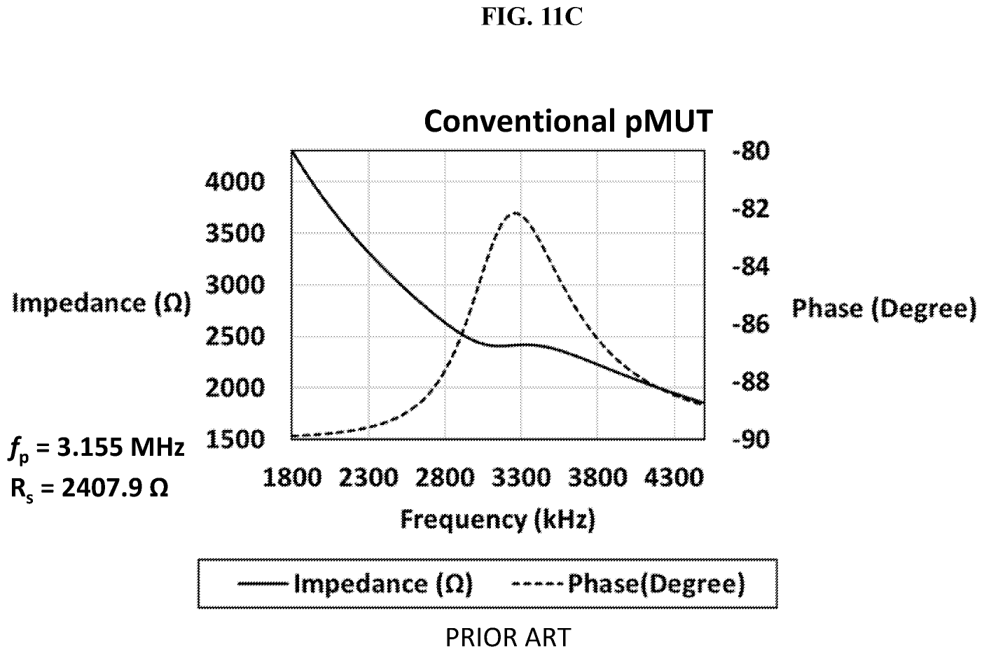

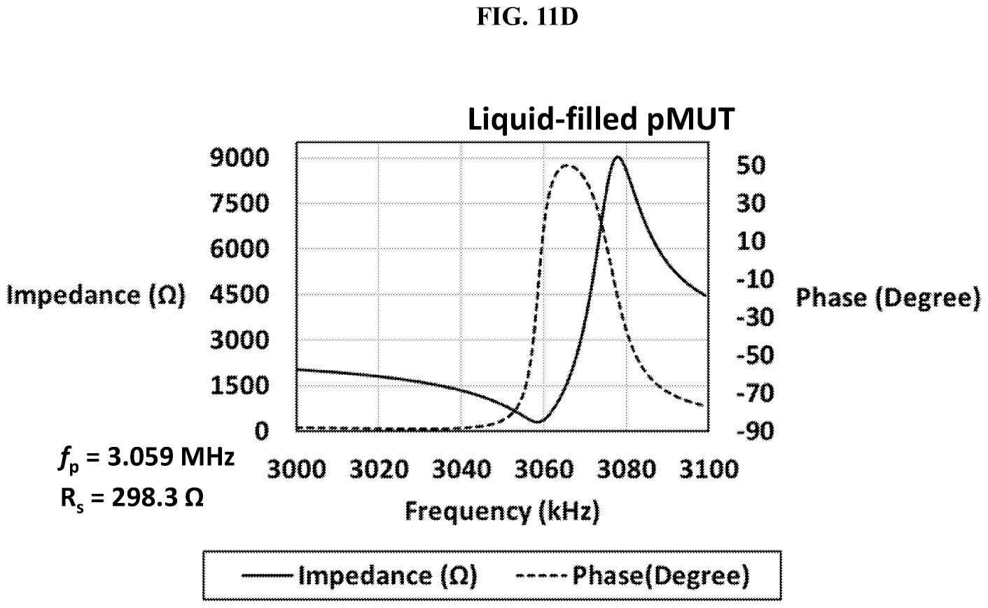

FIG. 11A is a plot of voltage (Voltage Mag, in millivolts or mV) as a function of frequency (freq, in Hertz or Hz) illustrating the receive sensitivity of the conventional piezoelectric micromachined acoustic transducer (pMUT). FIG. 11B is a plot of voltage (Voltage Mag, in millivolts or mV) as a function of frequency (freq, in Hertz or Hz) illustrating the receive sensitivity of the acoustic transducer with acoustic channel filled with a liquid according to various embodiments. FIG. 11C is a plot of impedance magnitude (in ohms or Ω)/impedance phase (in degrees) as a function of frequency (freq, in Hertz or Hz) illustrating the variation of the acoustic impedance magnitude and phase of the conventional piezoelectric micromachined acoustic transducer (pMUT). FIG. 11D is a plot of impedance magnitude (in ohms or Ω)/impedance phase (in degrees) as a function of frequency (freq, in Hertz or Hz) illustrating the variation of the acoustic impedance magnitude and phase of the acoustic transducer with acoustic channel filled with a liquid according to various embodiments.

As shown in FIGS. 11A-B, the receiving sensitivity (mV/kPa) was measured with the pressure 1 kPa applied on the pMUT surface. Due to the larger strain at resonant frequency ˜3 MHz, the receiving sensitivities will reach maximum value at this moment for both the conventional device and the liquid-filled device according to various embodiments. The peak value of receiving sensitivity is 86.9 mV/Pa for the liquid-filled device which is about 37 times higher than it of the conventional one due to its high Q factor. As shown in FIGS. 11C-D, the impedance magnitude and the corresponding phase are different for them, especially near the resonant frequency area. The series resonant impedance reduces by almost 8 times from 2407.89Ω for the conventional device to 298.31 Q for the liquid-filled device according to various embodiments, which is quite consistent with the law of energy conservation, where more electrical energy converts into dynamic energy.

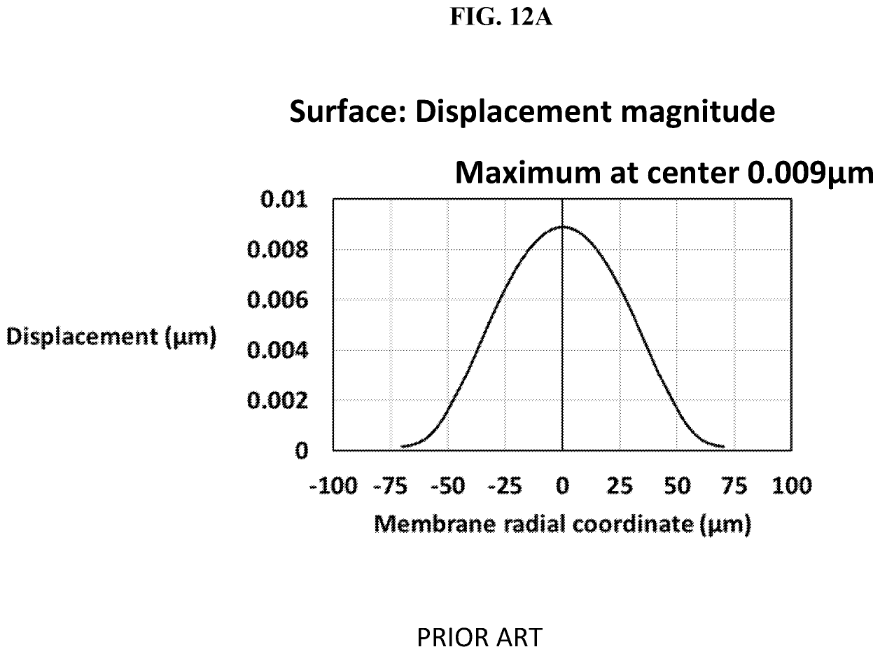

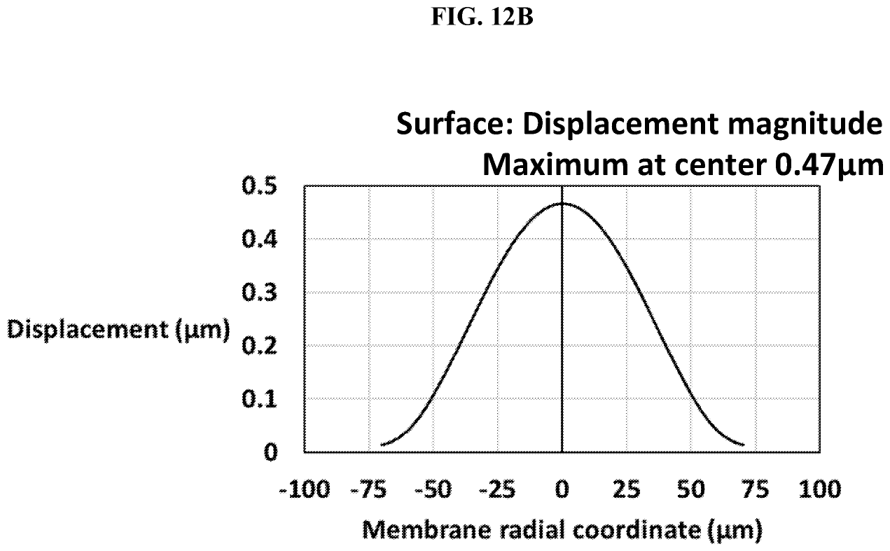

FIG. 12A shows a plot of displacement (in micrometers or μm) as a function of membrane radial coordinates (in micrometers or μm) illustrating the surface displacement magnitude for the conventional piezoelectric micromachined acoustic transducer (pMUT). FIG. 12B shows a plot of displacement (in micrometers or μm) as a function of membrane radial coordinates (in micrometers or μm) illustrating the surface displacement magnitude for the acoustic transducer with acoustic channel filled with a liquid according to various embodiments. The biggest displacement of the membrane may be obtained near the resonant frequency for both the conventional device and the liquid-filled device according to various embodiments. The deformation is increased by about 51 times from 9 nm for the conventional device to 465 nm for the liquid-filled device according to various embodiments. From the results of the liquid-filled device, it can be seen that the two resonant units (i.e. the transducer stack and the beam wall) may achieve a huge deformation at the resonant frequency. Each simulation results comparisons are coincident with each other.

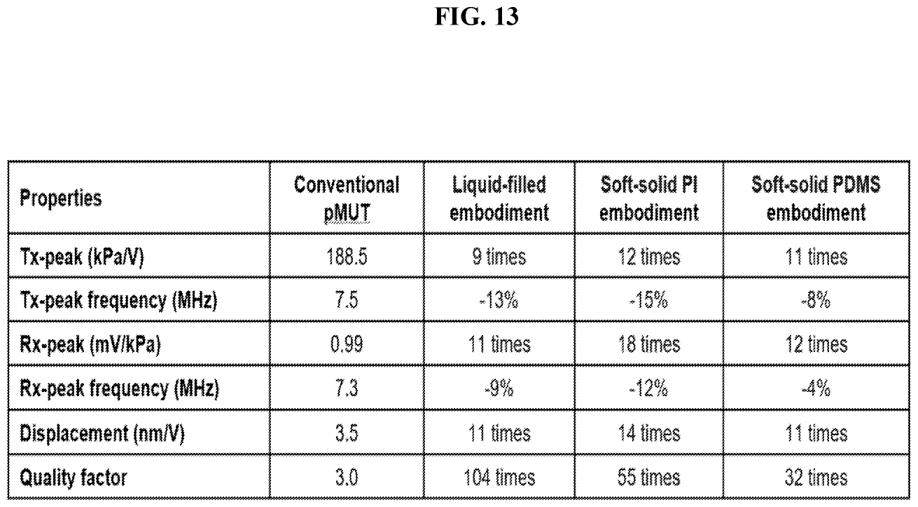

Simulation is also carried out on devices based on a ˜7 MHz pMUT with cavity of a radius 40 μm. A conventional device, the liquid-filled device according to various embodiments, the soft-solid PI device according to various embodiments, and the soft-solid PDMS device according to various embodiments may be simulated for verification, and the results are summarized in FIG. 13.

FIG. 13 is a table showing the simulation results of a conventional device, the liquid-filled device according to various embodiments, the soft-solid PI device according to various embodiments, and the soft-solid PDMS device according to various embodiments. Results of the devices with the acoustic channels are largely improved compared to the conventional device.

For the liquid-filled device, the optimized acoustic channel is designed with 50 μm radius, 10 μm width and 45 μm depth. However, for the soft-solid PI device and the soft-solid PDMS device, the optimized acoustic depth may be 50 μm and 30 μm respectively. Due to the different properties of the filled materials, the channel depth may need to be adjusted correspondingly to target a given frequency. This also can demonstrate that the acoustic medium may have a large influence on resonant frequency, with the boost in the pMUT vibration amplitude depending heavily on the coupled acoustic channel resonator.

Various embodiments may relate to a coupled MUT-resonant channel structure containing: (1) a micromachined ultrasonic transducer (MUT) unit with a targeted resonant frequency f_{MUT} and the corresponding 6 db bandwidth BW_{MUT} in a specific acoustic medium; (2) a resonant beam wall supporting the MUT that can vibrate in a flexural mode (optional); and (3) an adjacent acoustic channel with a certain width and depth filled with a liquid or low-stiffness solid, with stiffness lower than that of the MUT/substrate material. The acoustic channel may have an expected resonant frequency f_{Channel} and the corresponding 6 db bandwidth BW_{Channel}. There may also exist a strong coupling effect resulting in a big gain of Q factor (5˜300 times higher) and a big gain of output sensitivity (5˜100 times higher) effect when the MUT and the acoustic channel can meet the conditions: the relative frequency shift between f_{MUT} and f_{Channel} is no more than ±30% f_{MUT}, or the 6 dB bandwidth of MUT BW_{MUT} and the 6 dB bandwidth of acoustic channel BW_{Channel} can overlap more than 80%. The final gain and the coupling effect may be influenced by the relative frequency shift, the overlap of bandwidth and their own Q factors in specific acoustic medium. One effective way to optimize the gain to the maximum is to keep f_{MUT} constant and adjust the depth of acoustic channel to make f_{Channel} close to f_{MUT}.

The transducer stack may have a (horizontal) cross-sectional shape of any suitable shape.

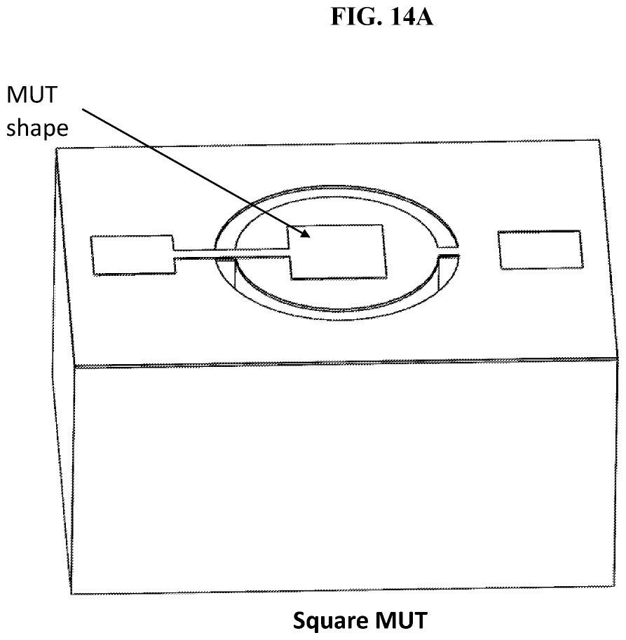

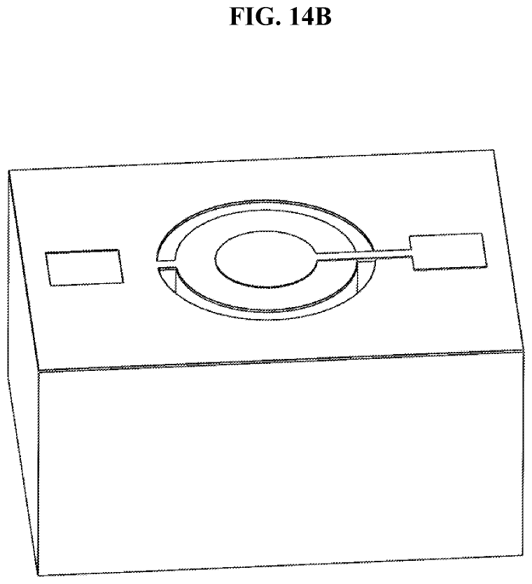

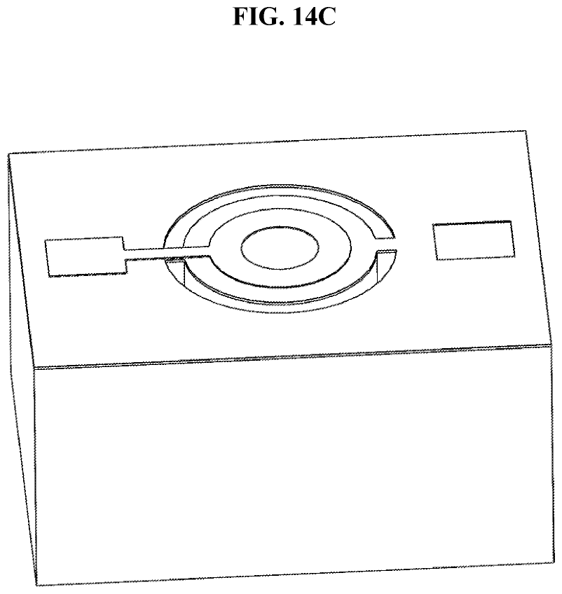

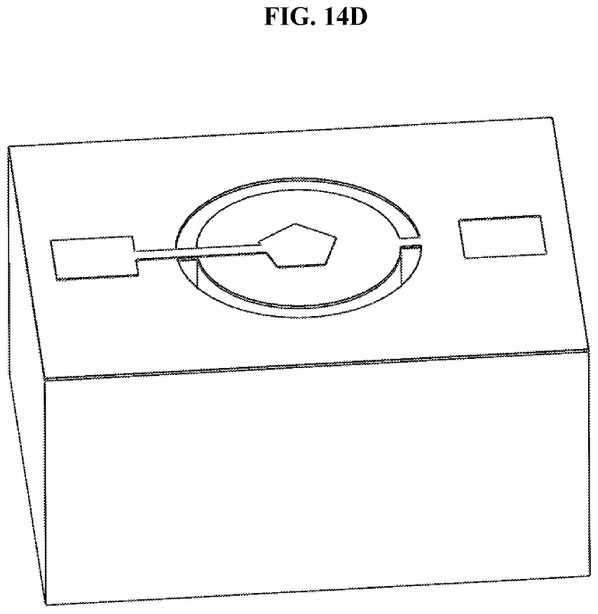

FIG. 14A shows an acoustic transducer having a square horizontal cross-sectional shape according to various embodiments. FIG. 14B shows an acoustic transducer having a circular horizontal cross-sectional shape according to various embodiments. FIG. 14C shows an acoustic transducer having an annular horizontal cross-sectional shape according to various embodiments. FIG. 14D shows an acoustic transducer having a pentagonal horizontal cross-sectional shape according to various embodiments.

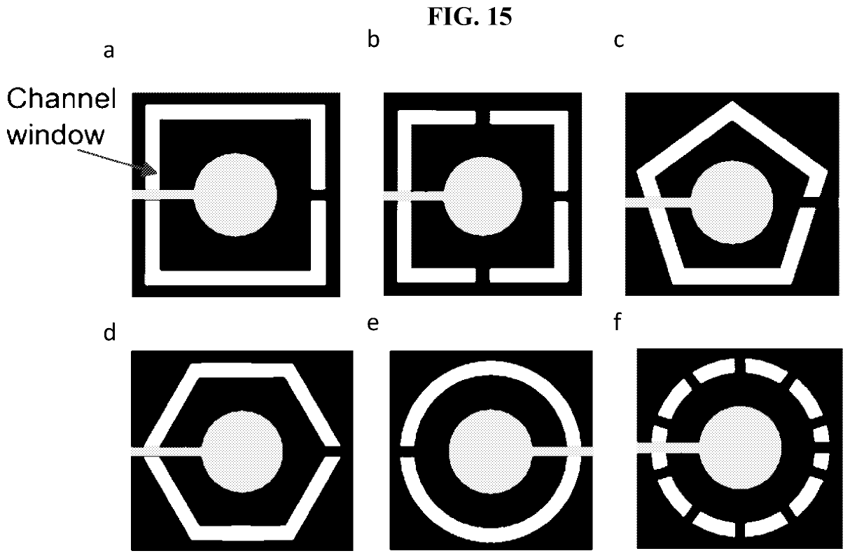

In various embodiments, the one or more acoustic channels may form a channel window of any suitable shape. FIG. 15 shows (a) an acoustic transducer having a continuous acoustic channel forming a square shape according to various embodiments; (b) an acoustic transducer having a discontinuous acoustic channel (i.e. a plurality of acoustic channels) forming a square shape according to various embodiments; (c) an acoustic transducer having a continuous acoustic channel forming a pentagonal shape according to various embodiments; (d) an acoustic transducer having a continuous acoustic channel forming a hexagonal shape according to various embodiments; (e) an acoustic transducer having a continuous acoustic channel forming a circular shape according to various embodiments; and (f) an acoustic transducer having a discontinuous acoustic channel (i.e. a plurality of acoustic channels) forming a circular shape according to various embodiments.

Various embodiments may have an ultimate transmit function much larger than a conventional acoustic transducer (i.e. one with only the transducer stack).

In various embodiments, the gain factor n and the amplification properties (gain factor and effective action frequency range) may be adjusted by the channel structure and channel filled with acoustic medium.

As highlighted above, the one or more acoustic channels may be configured to provide amplification of the initial signal, i.e. provide resonant channel amplification. In contrast, isolation features and impedance matching layer may not provide amplification of the initial signal.

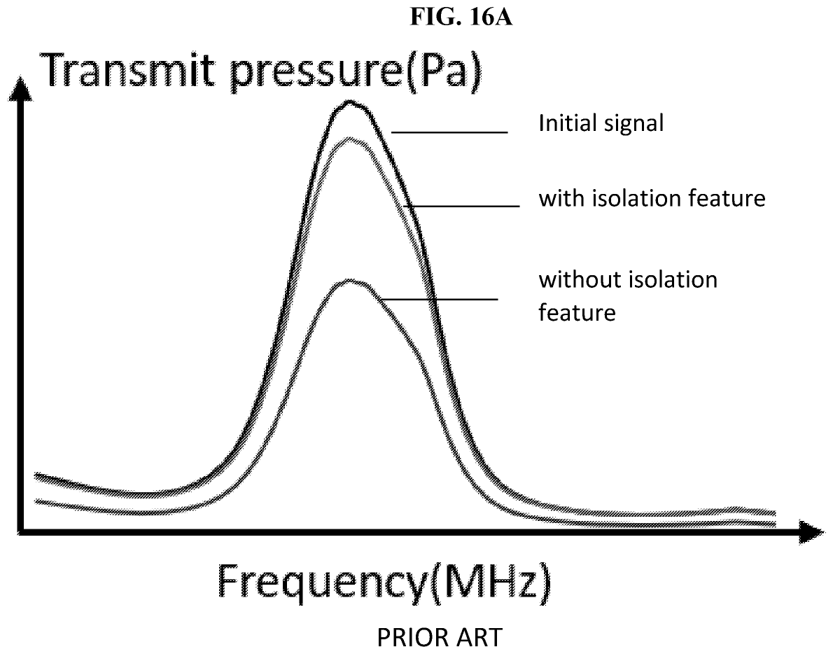

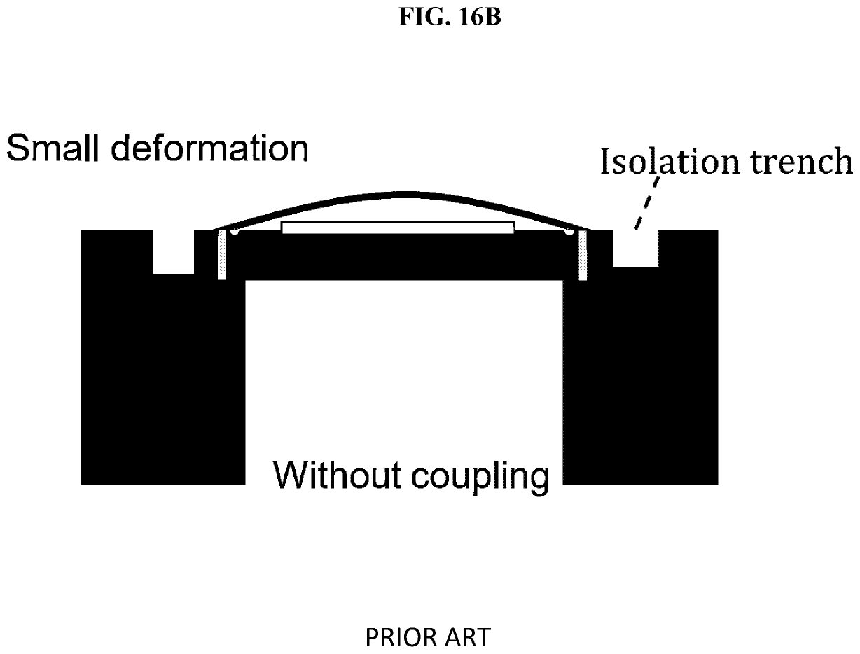

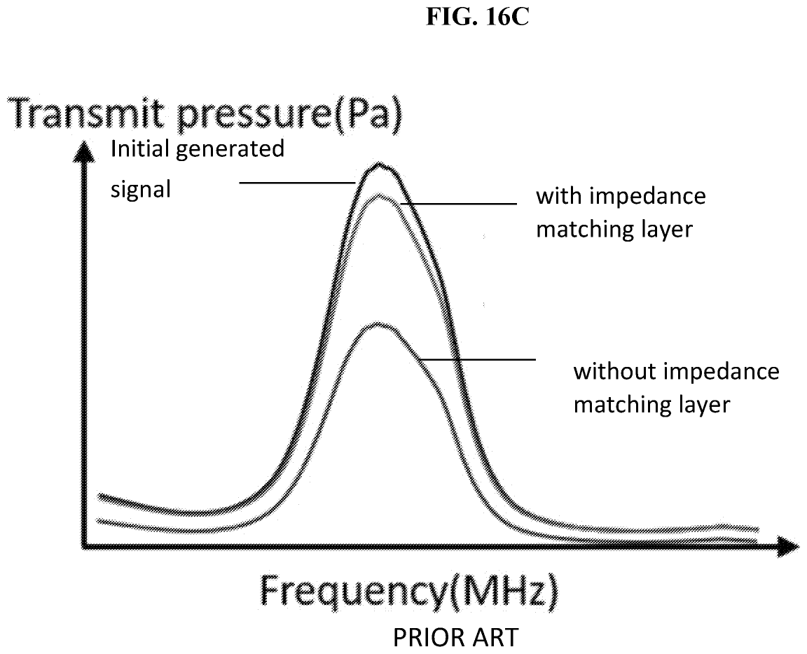

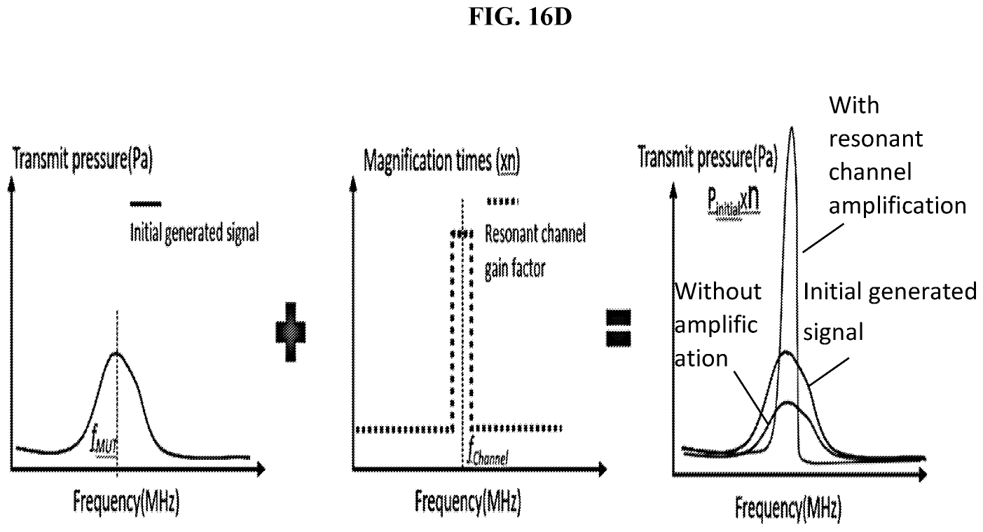

FIG. 16A is a plot of transmit pressure (in Pascals or Pa) as a function of frequency (in megahertz or MHz) showing the initial signal, the output signal with the isolation feature and the output signal without the isolation feature. FIG. 16B is a schematic showing an acoustic transducer with an isolation trench. FIG. 16C is a plot of transmit pressure (in Pascals or Pa) as a function of frequency (in megahertz or MHz) showing the initial signal, the output signal with the impedance matching layer and the output signal without the impedance matching layer. FIG. 16D shows (left) a plot of transmit pressure (in Pascals or Pa) as a function of frequency (in megahertz or MHz) showing the initial signal; (middle) a plot of magnification as a function of frequency (in megahertz or MHz) showing the resonant channel gain factor according to various embodiments; and (right) a plot of transmit pressure (in Pascals or Pa) as a function of frequency (in megahertz or MHz) showing the initial signal, the output signal with resonant channel amplification according to various embodiments and the output signal without resonant channel amplification.

It can be seen from FIGS. 16A-D that impedance matching and the isolation feature merely reduce dissipation of the initial generated signal, while resonant channel amplification provide amplification to the initial generated signal.

In various embodiments, the transducer stack may have a low Q factor. In various embodiments, the Q factor of the acoustic factor may be boosted by coupling the low Q factor transducer stack with a high Q factor acoustic channel, resulting in a large gain in transmit and receive sensitivities without enlarging the size of the acoustic transducer.

The one or more acoustic channels may be configured for channel resonance. For instance, each channel may be of a specific depth and with to create channel resonance, in which a strong resonance peak may be obtained. Each channel may be filled with an acoustic medium with a certain acoustic impedance to get sufficiently high incident energy from the transducer stack for coupling. The gains may be adjusted in different coupling conditions.

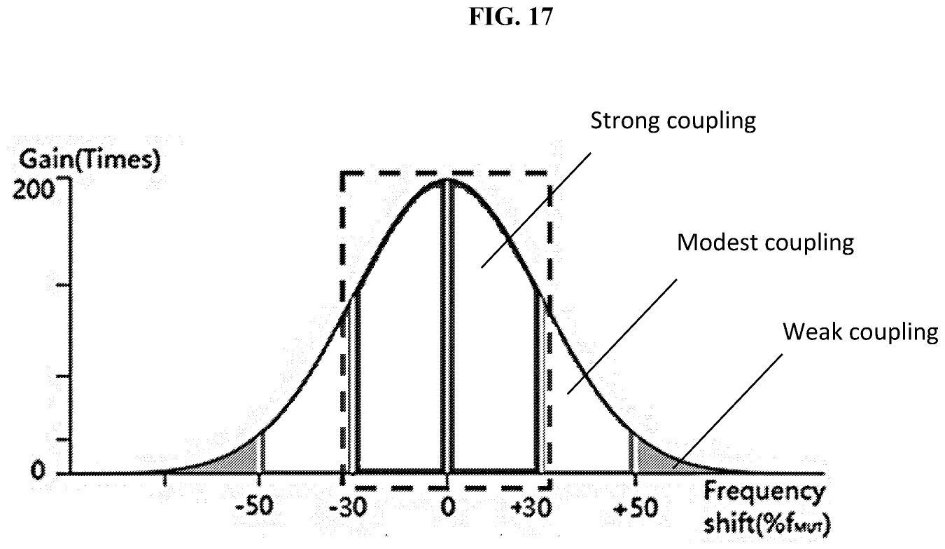

FIG. 17 is a plot of gain as a function of relative frequency shift (as a percentage (%) of the resonant frequency of the transducer unit) between the resonant frequency of the transducer unit of the acoustic transducer and the resonant frequency of the one or more acoustic channels of the acoustic transducer according to various embodiments.

The acoustic transducer may have strong coupling effect gains when a relative frequency shift between a resonant frequency of the transducer unit (f_{MUT}) and a resonant frequency of the one or more acoustic channels (f_{Channel}) is within 30% (i.e. ±30%) of the resonant frequency of the transducer unit (f_{MUT}), or where a 6 dB bandwidth of the ultrasonic transducer unit BW_{MUT} and a 6 dB bandwidth of the one or more acoustic channels BW_{Channel} overlap more than 80%. The strong coupling effect may result in a big gain of Q factor (2-300 times higher) and a big gain of output sensitivity (2-100 times higher)

The acoustic transducer may have modest coupling effect gains when a relative frequency shift between a resonant frequency of the transducer unit (f_{MUT}) and a resonant frequency of the one or more acoustic channels (f_{Channel}) is more than 30% (i.e. ±30%) but within 60% (i.e. ±60%) of the resonant frequency of the transducer unit (f_{MUT}), or where a 6 dB bandwidth of the ultrasonic transducer unit BW_{MUT} and a 6 dB bandwidth of the one or more acoustic channels BW_{Channel} overlap more than 30% but less than 80%. The modest coupling effect may result in a moderate gain of Q factor (1-10 times higher) and a moderate gain of output sensitivity (1-10 times higher).

Various embodiments may relate to a micromachined ultrasonic transducer (MUT) structure. The structure may be distinguished from the conventional single MUT in that it contains multiple resonant units. Each of the resonant units may have its own eigen frequency. When the adjacent resonant units fit well with each other or one another, it may result in a big coupling effect resulting in a comprehensive output pressure. The channel resonator may play a huge role to adjust this coupling relationship. The structural parameters (e.g. depth and/or width) and material properties may be required to be set properly to match the MUT unit resonant frequency and achieve sufficient high coupling energy.

In various embodiments, the resonant units may include the beam wall in addition to the transducer unit, and the one or more acoustic channels with the acoustic medium. The transmit and receive sensitivities may be enhanced largely by the coupling of each part. The structural design may help reduce stress and increase the vibration area, and may prevent energy dissipation into the substrate. In particular, there may be a coupling effect between the low Q transducer unit and the adjacent high Q channel resonator. There may be a strong coupling effect, resulting in a big transmit (TX) gain (2-100 times) or a modest coupling effect, resulting in a moderate TX gain (1-10 times) in water/air. In various embodiments, the transmit sensitivity may have an enhancement of greater than 5 to 100 times (compared to a conventional acoustic transducer). In various embodiments, the receive sensitivity may have an enhancement of greater than 5 to 100 times. In various embodiments, the quality factor may have an enhancement of greater than 5 to 300 times. There may be huge performance gain achieved by liquid-filled acoustic channel optimization or soft-solid material filled acoustic channel optimization. Various embodiments may be difficult to bypass, especially for achieving the big transmit/Q factor performance gain (>10 times). In one example, the transmit sensitivity may increase by 46 times, the receive sensitivity may increase by 37 times, the Q factor may increase by 209 times, and the displacement may increase by 51 times.

Various embodiments may provide enhancement of MUT output and receive sensitivities by coupling to the adjacent high-Q resonant systems to boost effective quality factor. Various embodiments may be used for energy delivery or reception applications with a small form factor. Various embodiments may not be for imaging applications that require low Q factor.

Various embodiments may be useful for ultrasonic applications that require a high output pressure sensitivity (kPa/V) or high receive sensitivity (mV/kPa) with ultrasonic frequency range (˜1 MHz) and small package size (˜mm). Various embodiments may be an energy delivery pMUT, which provides high output pressure for high energy delivery, and which may have a small form factor. The energy delivery pMUT may be used in applications that require high acoustic output energy and small form factor, such as dental cleaning or medical ultrasonic therapy.

1. An acoustic transducer comprising:
a substrate comprising a cavity extending from a surface of the substrate, wherein the substrate further comprises one or more acoustic channels, each of the one or more acoustic channels having a depth extending from the surface of the substrate and a length, the length greater than the depth, extending at least partially around the cavity; and
a layered arrangement suspended over the cavity;
wherein the one or more acoustic channels are configured to comprise an acoustic medium having a specific acoustic impedance lower than a specific acoustic impedance of the substrate.

2. The acoustic transducer according to claim 1, wherein the one or more acoustic channels comprising the acoustic medium provide amplification to an initial acoustic transmit signal generated by the membrane stack upon application of the voltage to the transducer stack.

3. The acoustic transducer according to claim 1, wherein the one or more acoustic channels comprising the acoustic medium provide amplification to an initial electrical signal generated by the transducer stack upon the ultrasound wave incident onto the membrane stack.

4. The acoustic transducer according to claim 1, wherein the substrate comprises a beam wall to at least partially define the cavity and the one or more acoustic channels, the beam wall separating the cavity from the one or more acoustic channels.

5. The acoustic transducer according to claim 4, wherein an end portion of the beam wall is in contact with the membrane stack.

6. The acoustic transducer according to claim 4, wherein the beam wall is configured to vibrate in a flexural mode upon application of an excitation voltage.

7. The acoustic transducer according to claim 1, wherein the acoustic medium is a solid.

8. The acoustic transducer according to claim 7, wherein the solid is polyimide or polydimethylsiloxane.

9. The acoustic transducer according to claim 1, wherein the acoustic medium is a liquid.

10. The acoustic transducer according to claim 9, wherein the liquid is water or alcohol.

11. The acoustic transducer according to claim 1, wherein an effective stiffness of the acoustic medium is lower than an effective stiffness of the layered arrangement and an effective stiffness of the substrate.

12. The acoustic transducer according to claim 1, wherein the depth of each of the one or more acoustic channels further extends through the layered arrangement.

13. The acoustic transducer according to claim 1, wherein the acoustic transducer is a piezoelectric micromachined acoustic transducer or a capacitive micromachined acoustic transducer.

14. The acoustic transducer according to claim 1,
wherein the layered arrangement comprises a transducer stack; and
wherein the transducer stack has a cross-sectional shape selected from a group consisting of a square, a circle, an annulus, a polygon, and an ellipse.

15. The acoustic transducer according to claim 1, wherein the one or more acoustic channels form a shape selected from a group consisting of a square, a circle, a pentagon, and a hexagon.

16. The acoustic transducer according to claim 1,
wherein the layered arrangement comprises a transducer stack; and
wherein the transducer stack comprises:
a first electrode;
a second electrode; and
a piezoelectric layer between the first electrode and the second electrode.

17. The acoustic transducer according to claim 1, wherein the layered arrangement comprises:
a first dielectric layer;
a second dielectric layer; and
a membrane layer between the first dielectric layer and the second dielectric layer.

18. The acoustic transducer according to claim 1,
wherein a relative frequency shift between a resonant frequency of a transducer unit, the transducer unit including the layered arrangement and the cavity, and a resonant frequency of the one or more acoustic channels is within 30% of the resonant frequency of the transducer unit; or
wherein a 6 dB bandwidth of the transducer unit and a 6 dB bandwidth of the one or more acoustic channels overlap more than 80%.

19. The acoustic transducer according to claim 1,
wherein a relative frequency shift between a resonant frequency of a transducer unit, the transducer unit including the layered arrangement and the cavity, and a resonant frequency of the one or more acoustic channels is more than 30% but within 60% of the resonant frequency of the transducer unit; or
wherein a 6 dB bandwidth of the transducer unit and a 6 dB bandwidth of the one or more acoustic channels overlap more than 30% but less than 80%.

20. A method of forming an acoustic transducer, the method comprising:
patterning a substrate such that the substrate comprises a cavity extending from a surface of the substrate, wherein the substrate further comprises one or more acoustic channels, each of the one or more acoustic channels having a depth extending from the surface of the substrate and a length, the length greater than the depth, extending at least partially around the cavity; and
forming a layered arrangement suspended over the cavity;
wherein the one or more acoustic channels are configured to comprise an acoustic medium having a specific acoustic impedance lower than a specific acoustic impedance of the substrate.
